(19) United States
(12) Patent Application Publication
(10) Pub. No.: US 2016/0122758 A1
(43) Pub. Date: May 5, 2016

(54) AGENTS FOR DOWNREGULATION OF THE ACTIVITY AND/OR AMOUNT OF BCL-XL AND/OR BCL-W

(71) Applicant: YEDA RESEARCH AND DEVELOPMENT CO. LTD., Rehovot (IL)

(72) Inventors: Valery KRIZHANOVSKY, Rehovot (IL); Noam PILPEL, Rehovot (IL); Reut YOSEF, Rehovot (IL)

(21) Appl. No.: 14/784,312

(22) PCT Filed: Apr. 13, 2014
(86) PCT No.: PCT/IL2014/050358

§ 371 Date: Oct. 14, 2015

Related U.S. Application Data
(60) Provisional application No. 61/814,286, filed on Apr. 21, 2013.

(51) Int. Cl. C12N 15/113; A61K 9/00; A61K 31/635; A61K 45/06; A61K 38/17

(52) U.S. Cl. CPC C12N 15/113; A61K 45/06; A61K 38/1709; A61K 31/635; A61K 9/0014; C12N 2310/14; C12N 2320/31

(57) ABSTRACT

A method of treating an inflammatory or fibrotic disease in a subject is disclosed. The method comprises administering to the subject a therapeutically effective amount of an agent which down-regulates an activity and/or an amount of Bcl-xL and/or Bcl-w and/or p21, with the proviso that the inflammatory disease is not cancer.

### FIELD AND BACKGROUND OF THE INVENTION

The present invention, in some embodiments thereof, relates to a method of killing senescent cells by the down-regulation of genes encoding Bcl-2-family proteins and/or p21 for the treatment of age-related disorders.

Cellular senescence, a stable form of cell cycle arrest, is a mechanism limiting the proliferative potential of cells. Senescence can be triggered in many cell types in response to diverse forms of cellular stress. It is a potent barrier to tumorigenesis and contributes to the cytotoxicity of certain anti-cancer agents. While senescence limits tumorigenesis and tissue damage in a cell autonomous manner, senescent cells induce inflammation, tissue ageing, tissue destruction and promote tumorigenesis and metastasis in a cell non-autonomous manner in the sites of their presence. Therefore, their elimination might lead to tumor prevention and inhibition of tissue ageing. Indeed, elimination of senescent cells was shown to slow down tissue ageing in an animal model (Baker et al., 2011).

Organisms might have developed elaborate mechanisms to eliminate senescent cells in order to avoid their deleterious effects on the microenvironment. However, their fate in tissue is not well characterized. On one hand, benign melanocytic nevi (moles) are highly enriched for senescent cells yet can exist in skin throughout a lifetime, implying that senescent cells can be stably incorporated into tissues. On the other hand, it has been previously shown that components of the innate immune system specifically recognize and eliminate senescent cells in vitro and target senescent cells in vivo leading to tumor regression and reversion of liver fibrosis (Krizhanovsky et al., 2008b; Sagiv et al., 2012; Xue et al., 2007). Therefore, senescent cells can turn over in vivo and the immune system contributes to this turnover. The effort that the immune system invests in recognition and elimination of senescent cells suggests, although not directly, that senescent cells are deleterious for the organism and their elimination is beneficial.

In the last decade multiple studies identified the genes and the pathways required for senescence induction or bypass of the senescence phenotype. Two tumor suppressor pathways, controlled by the p53 (TP53) and p16INK4a (CDKN2A), regulate senescence response. p53 promotes senescence by transactivating genes that inhibit proliferation, while p16INK4a, accompanied by the p53 target p21 (CDKN1A), inhibit cyclin-dependent kinases (CDKs) 2 and 4, thereby preventing pRB phosphorylation and promoting repressive heterochromatin formation to silence proliferation-associated genes.

Bcl-2-family proteins play a central role in cell death regulation and are capable of regulating diverse cell death mechanisms that encompass apoptosis, necrosis and autophagy (Cory et al., 2003; Reed, 2008). The function of the founding member of the family, Bcl-2, in senescence remains controversial. It was proposed to be either upregulated or downregulated in senescent cells and was associated with either negative or positive regulation of apoptosis of these cells (Uraoka et al., 2011; Wang, 1995). In addition to Bcl-2, the family includes the anti-apoptotic proteins Bcl-xL, Bcl-w, Mcl-1 and A1, and is intensively studied as a target for pharmacological intervention in cancer (Azmi et al., 2011; Zeitlin et al., 2008).

U.S. Patent Application No. 20120189539 teaches a chemical which down-regulates Bcl-xL for the treatment of cancer.

U.S. Patent Application No. 20040001811 teaches pharmaceutical compositions comprising dsRNA targeted against Bcl-2 family members for the treatment of cancer.

U.S. Patent Application No. 20070258952 teaches administration of siRNA targeted against numerous genes including Bcl-xL and p-21.

U.S. Patent Application No. 20110301192 teaches administration of chemical agents that down-regulate p-21 for the treatment of cancer.

### SUMMARY OF THE INVENTION

According to an aspect of some embodiments of the present invention there is provided a method of treating an inflammatory or fibrotic disease in a subject in need thereof comprising administering to the subject a therapeutically effective amount of an agent which down-regulates an activity and/or an amount of Bcl-xL and/or Bcl-w, thereby treating the inflammatory or fibrotic disease, with the proviso that the inflammatory disease is not cancer.

According to an aspect of some embodiments of the present invention there is provided an article of manufacture comprising:

(i) an agent which down-regulates an activity and/or an amount of Bcl-xL and/or Bcl-w; and

(ii) an agent which down-regulates an activity and/or an amount of p21.

According to an aspect of some embodiments of the present invention there is provided a pharmaceutical composition comprising a pharmaceutically acceptable carrier and as an active agent:

(i) an agent which down-regulates an activity and/or an amount of Bcl-xL and/or Bcl-w; and

(ii) an agent which down-regulates an activity and/or an amount of p21.

According to an aspect of some embodiments of the present invention there is provided an agent which down-regulates an activity and/or an amount of cyclin-dependent kinase inhibitor 1 (p21) for use in treating an inflammatory or fibrotic disease, wherein the disease is not cancer.

According to an aspect of some embodiments of the present invention there is provided a polynucleotide agent which down-regulates an endogenous nucleic acid sequence expressing Bcl-xL and a polynucleotide agent which down-regulates an endogenous nucleic acid sequence expressing Bcl-w for use in treating an inflammatory or fibrotic disease.

According to an aspect of some embodiments of the present invention there is provided a composition comprising a carrier and at least one active agent which down-regulates an activity and/or an amount of p21 and at least one active agent which down-regulates an activity and/or an amount of Bcl-xL and/or Bcl-w, wherein the composition is formulated for topical administration.

According to an aspect of some embodiments of the present invention there is provided a method of treating an inflammatory or fibrotic disease in a subject in need thereof comprising administering to the subject a therapeutically effective amount of an agent which down-regulates an activity and/or an amount of cyclin-dependent kinase inhibitor 1 (p21), thereby treating the inflammatory or fibrotic disease, with the proviso that the disease is not cancer.

According to an aspect of some embodiments of the present invention there is provided a method of treating an inflammatory or fibrotic disease in a subject in need thereof comprising administering to the subject a therapeutically effective amount of at least one polynucleotide agent which down-regulates an endogenous nucleic acid sequence expressing Bcl-xL and at least one polynucleotide agent which down-regulates an endogenous nucleic acid sequence expressing Bcl-w, thereby treating the inflammatory or fibrotic disease.

According to an aspect of some embodiments of the present invention there is provided a method of treating a pre-malignant lesion in a subject in need thereof comprising administering to the subject a therapeutically effective amount of an agent which down-regulates an activity and/or an amount of Bcl-xL and/or Bcl-w, thereby treating the pre-malignant lesion.

According to an aspect of some embodiments of the present invention there is provided a method of treating a pre-malignant lesion in a subject in need thereof comprising administering to the subject a therapeutically effective amount of an agent which down-regulates an activity and/or an amount of cyclin-dependent kinase inhibitor 1 (p21), thereby treating the pre-malignant lesion.

According to some embodiments of the invention, the agent is a chemical agent. According to some embodiments of the invention, the agent is a polynucleotide agent targeted against the Bcl-xL and/or Bcl-w.

According to some embodiments of the invention, the disease is associated with cartilage degeneration.

According to some embodiments of the invention, the disease is selected from the group consisting of liver fibrosis, wound healing, skin fibrosis, pulmonary disease, osteoporosis, kidney fibrosis, prostatitis, atherosclerosis, arthritis and pancreatitis.

According to some embodiments of the invention, the pulmonary disease comprises chronic obstructive pulmonary disease (COPD).

According to some embodiments of the invention, the agent which down-regulates regulates an activity and/or an amount of Bcl-xL and/or Bcl-w is comprised in a separate packaging to the agent which down-regulates an activity and/or an amount of p21.

According to some embodiments of the invention, the agent which down-regulates regulates an activity and/or an amount of Bcl-xL and/or Bcl-w is comprised in the same packaging as the agent which down-regulates an activity and/or an amount of p21.

According to some embodiments of the invention, the article of manufacture further comprises at least one agent selected from the group consisting of a sebum-regulating agent, an antibacterial and/or antifungal agent, a keratolytic agent and/or keratoregulating agent, an astringent, an anti-inflammatory and/or anti-irritant, an antioxidant and/or free-radical scavenger, a cicatrizing agent, an anti-aging agent and a moisturizing agent.

According to some embodiments of the invention, the at least one agent is an anti-aging agent.

According to some embodiments of the invention, the pharmaceutical composition is formulated for topical delivery.

According to some embodiments of the invention, the polynucleotide agent is an siRNA agent.

According to some embodiments of the invention, the at least one active agent which down-regulates an activity and/or an amount of Bcl-xL and/or Bcl-w is ABT-737 or ABT-263.

According to some embodiments of the invention, the composition further comprises at least one agent selected from the group consisting of a sebum-regulating agent, an antibacterial and/or antifungal agent, a keratolytic agent and/or keratoregulating agent, an astringent, an anti-inflammatory and/or anti-irritant, an antioxidant and/or free-radical scavenger, a cicatrizing agent, an anti-aging agent and a moisturizing agent.

According to some embodiments of the invention, the at least one agent is an anti-aging agent.

According to some embodiments of the invention, the agent is a polynucleotide directed to an endogenous nucleic acid sequence expressing the p21.

According to some embodiments of the invention, the polynucleotide agent is an siRNA.

According to some embodiments of the invention, the method further comprises administering to the subject at least one agent which down-regulates an activity and/or an amount of Bcl-xL and/or Bcl-w.

According to some embodiments of the invention, the at least one agent is a polynucleotide directed to an endogenous nucleic acid sequence expressing the Bcl-xL and/or Bcl-w.

According to some embodiments of the invention, the agent is an siRNA directed against Bcl-xL and/or Bcl-w.

According to some embodiments of the invention, the at least one agent is a chemical agent.

According to some embodiments of the invention, the chemical agent is selected from the group consisting of ABT-737, ABT-263, Gossypol, AT-101, TW-37 and Obatoclax.

According to some embodiments of the invention, the disease is associated with cartilage degeneration.

According to some embodiments of the invention, the disease is selected from the group consisting of liver fibrosis, wound healing, skin fibrosis, pulmonary disease, kidney fibrosis, prostatitis, atherosclerosis, arthritis and pancreatitis.

According to some embodiments of the invention, the agent is formulated as a topical composition.

According to some embodiments of the invention, the at least one polynucleotide agent comprises an siRNA.

According to some embodiments of the invention, the disease is cancer.

According to some embodiments of the invention, the disease is selected from the group consisting of liver fibrosis, wound healing, skin fibrosis, pulmonary disease, kidney fibrosis, prostatitis, atherosclerosis, arthritis and pancreatitis.

According to some embodiments of the invention, the at least one agent is formulated as a topical composition.

According to some embodiments of the invention, the method further comprises administering to the subject an agent which down-regulates an activity and/or an amount of p21.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings and images. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-C illustrate the elevated expression of Bcl-w and Bcl-xL proteins in senescent cells. (A) Immunoblots of cellular lysates (IMR-90 and MEF) corresponding to vehicle treated growing cells (G), cells treated with Etoposide to induce senescence (Eto); cells transduced with empty vector (V) or with H-ras^{v12} expressing (H-ras^{v12}) retroviruses. β-tubulin served as a loading control. (B) SA-β-gal activity staining performed on IMR-90 cells treated as described in A. (C) Quantitative RT-PCR analysis of mRNA levels of Bcl-2, Bcl-w and Bcl-xL in IMR-90 cells treated as described in A. Values are mean+SEM.

FIGS. 2A-B illustrate that combined knockdown of Bcl-w and Bcl-xL induces killing of senescent cells. (A) Etoposide treated senescent IMR-90 cells were transfected with the indicated siRNAs. Cell viability was determined four days post transfection. (B) Western blot analysis for Bcl-2, Bcl-w and Bcl-xL expression at four days after transfection of Etoposide treated senescent cells with the indicated siRNAs. β-tubulin served as a loading control.

FIGS. 3A-D illustrate that combined knockdown of Bcl-w and Bcl-xL induces senescent cell death. (A) Etoposide treated senescent IMR-90 cells were transfected with the indicated siRNAs. Cell viability was determined four days post transfection. (B) Etoposide treated senescent IMR-90 cells were transfected with the indicated siRNAs. Three days post transfection cells were treated with 10 μM ABT-199 for 24 hours or with DMSO as control. Cell viability was determined at the end of the incubation time. (C) IMR-90 or MEF growing vehicle treated cells (G), senescent Etoposide treated cells (E), cells transduced with empty vector (V) or with H-ras^{V12} expressing (H-ras^{V12}) retroviruses were treated with ABT-199 for 24 hours. Cell viability was determined at the end of the incubation time. SH-SY5Y cells, which are sensitive to Bcl-2 inhibition, were used as positive control for treatment.

FIGS. 4A-B illustrate that the BH3 mimetic ABT-737 induces cell death in senescent cells. IMR-90 (FIG. 4A) or MEF (FIG. 4B) growing cells (G), cells treated with Etoposide (Eto), cells transduced with empty vector (V) or with H-ras^{V12} expressing (H-ras^{V12}) retroviruses were treated with the indicated doses of ABT-737 or with DMSO as control for 24 hours. Cell viability was determined at the end of the incubation time.

FIGS. 5A-B illustrate IMR-90 growing cells (G), cells treated with Etoposide (Eto) or cells transduced with H-ras^{v12} (Ras) expressing retroviruses treated with ABT-737 or DMSO as control for 24 hours in the presence or absence of z-VAD-fmk. (A) Cell viability was determined at the end of the incubation time. (B) Immunoblots of cellular lysates corresponding to growing cells (G), Etoposide treated cells (Eto) or cells transduced with H-ras^{v12} (Ras) expressing retroviruses in the presence of DMSO, ABT-737 or ABT-737 plus z-VAD-fmk as indicated. β-tubulin served as a loading control.

FIGS. 6A-E illustrate that p21 affects the viability of senescent cells. (A-D) Growing (G) and Etoposide (Eto) treated primary human (IMR-90, BJ) and mouse (MEF) fibroblasts as well as human lung cancer cells (H1299) were transfected with the siRNA against p21 or control siRNA. Cell viability was determined four days post transfection (A-D) or at the indicated time points (E).

FIGS. 7A-C illustrates that p21 maintains the viability of senescent cells in p53 and pRB independent manner. (A) Western blot analysis for the indicated proteins at four days after transfection of growing (G) and Etoposide treated BJ cells (Eto) with siRNA for p21 or control siRNA. β-tubulin served as a loading control. (B-C) Etoposide treated BJ cells were transfected with the indicated siRNA. Cell viability was determined four days post transfection. In the same time, cell lysates were collected and analyzed by western blots to verify efficient knockdown of the indicated protein.

FIG. 8 is a graph illustrating that inhibition of Caspase activity partially rescues senescent cells from apoptosis. Etoposide treated BJ cells were transfected with sip21 or control siRNA. Cells were incubated for four days in the presence or absence of z-VAD-fmk (with daily replenishment). Cell viability was determined at the end of the incubation time.

FIGS. 9A-E illustrate that E2F targets and inflammation genes are upregulated as a response to p21 knockdown. Quantitative RT-PCR analysis of mRNA levels of growing (G) and Etoposide (Eto) treated BJ cells for the indicated genes. GAPDH mRNA was used as a reference. Data expressed as average+SEM of 3 independent RT-PCR analyses.

FIG. 10 is a photograph illustrating that the presence of both senescent cells and the fibrotic scar was diminished in p21 knockout mice. Wild type and p21−/− (knockout) mice were subjected to a six week treatment with CCl₄ to induce fibrosis. Following the treatment livers were evaluated by SA-β-gal for presence of senescent cells and by Sirius Red staining for fibrotic scars formation.

### DESCRIPTION OF SPECIFIC EMBODIMENTS OF THE INVENTION

The present invention, in some embodiments thereof, relates to a method of killing senescent cells by the down-regulation of genes encoding Bcl-2-family proteins and/or p21 for the treatment of age-related disorders.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Senescent cells can be found in fibrotic or inflammatory diseases of skin, liver, lung, pancreas, prostate, as well as in articular cartilage, atherosclerotic plaques and other age-related diseases. Moreover, senescent cells were shown to accumulate in normal tissues, especially skin, with age and suggested to contribute to tissue ageing. Therefore, elimination of senescent cells might significantly delay ageing of many tissues and treat the pathological conditions where senescent cells are present.

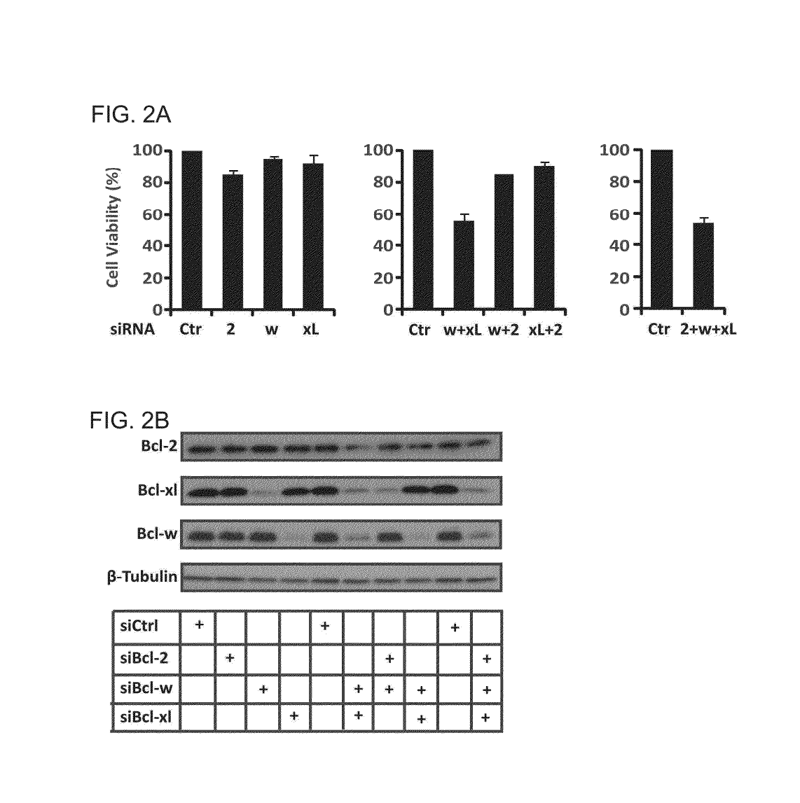

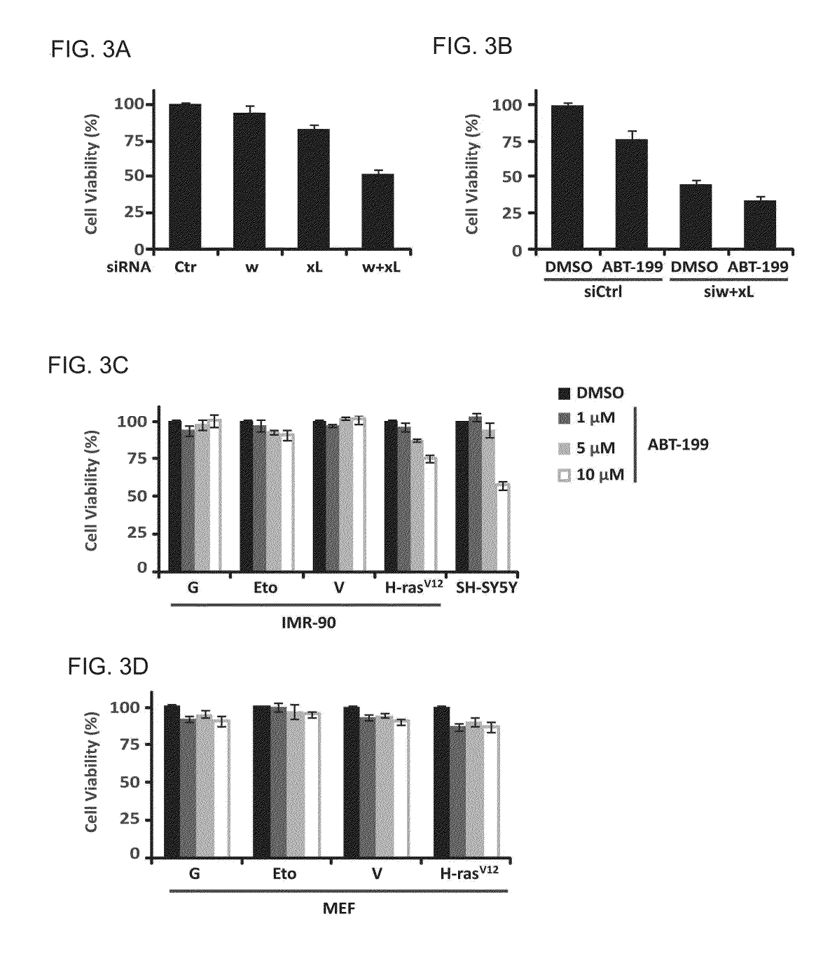

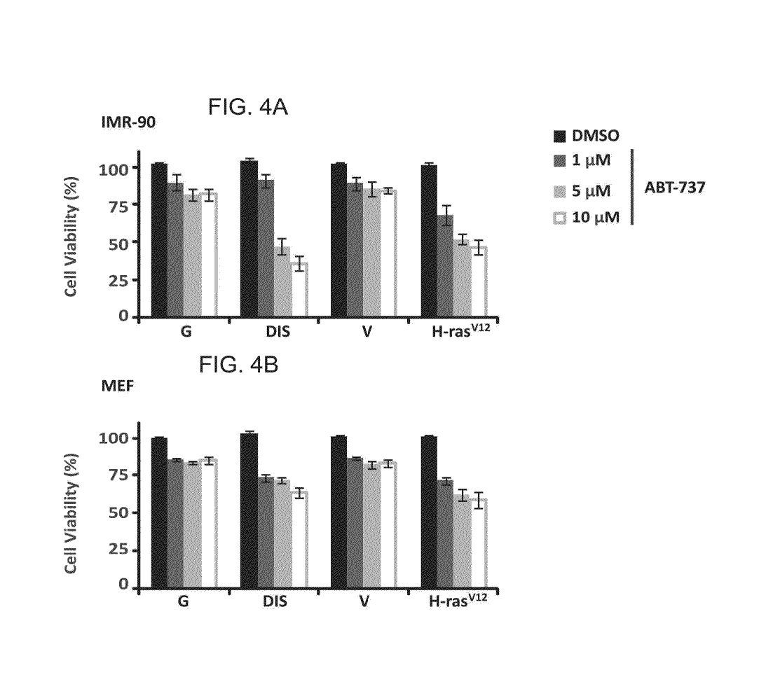

The present inventors have shown that combined inhibition of Bcl-xL and Bcl-w either by siRNA (FIGS. 2A-B and 3A-B) or by a specific inhibitor of the Bcl-2 family, (ABT-737; FIGS. 4A-B and 5A-B) leads to specific elimination of senescent cells. Inhibition of Bcl-2 itself fails to perform this task (FIGS. 3C-D). Accordingly, the present inventors propose that combined inhibition of Bcl-xL and Bcl-w allows specific elimination of senescent cells and may be used to treat diseases where senescent cells are present.

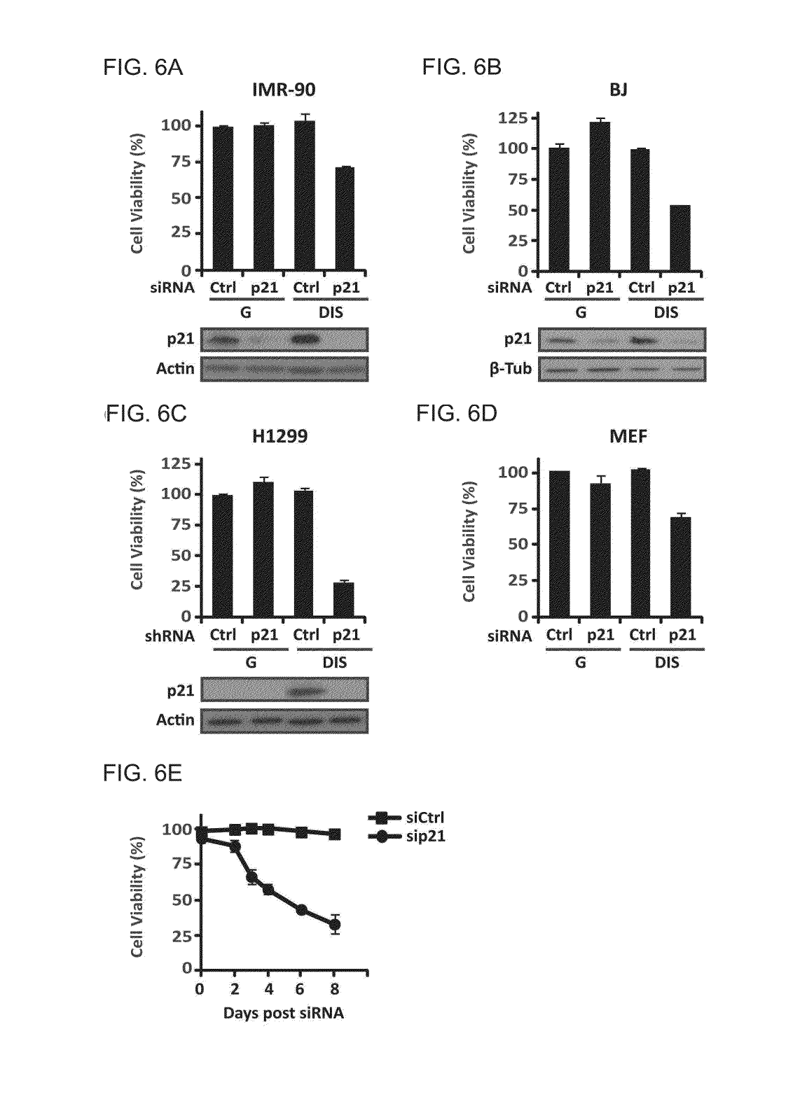

Surprisingly, the present inventors have discovered that the same effect on senescent cells may be achieved by reduction of the expression of p21 (FIGS. 6A-E and 7A-C), a protein typically associated with the onset of senescence (as an inhibitor of CDK4 and CDK2) and tumor suppression. Whereas p21 knockdown in growing cells had no deleterious effect on cell viability, its knockdown in senescent cells led to a 30%, 50%, 75% and 30% reduction in cell viability for IMR-90, BJ, H1299 and MEF cells respectively (FIG. 6A). Thus, the present inventors propose that p21 is necessary to maintain the viability of senescent cells.

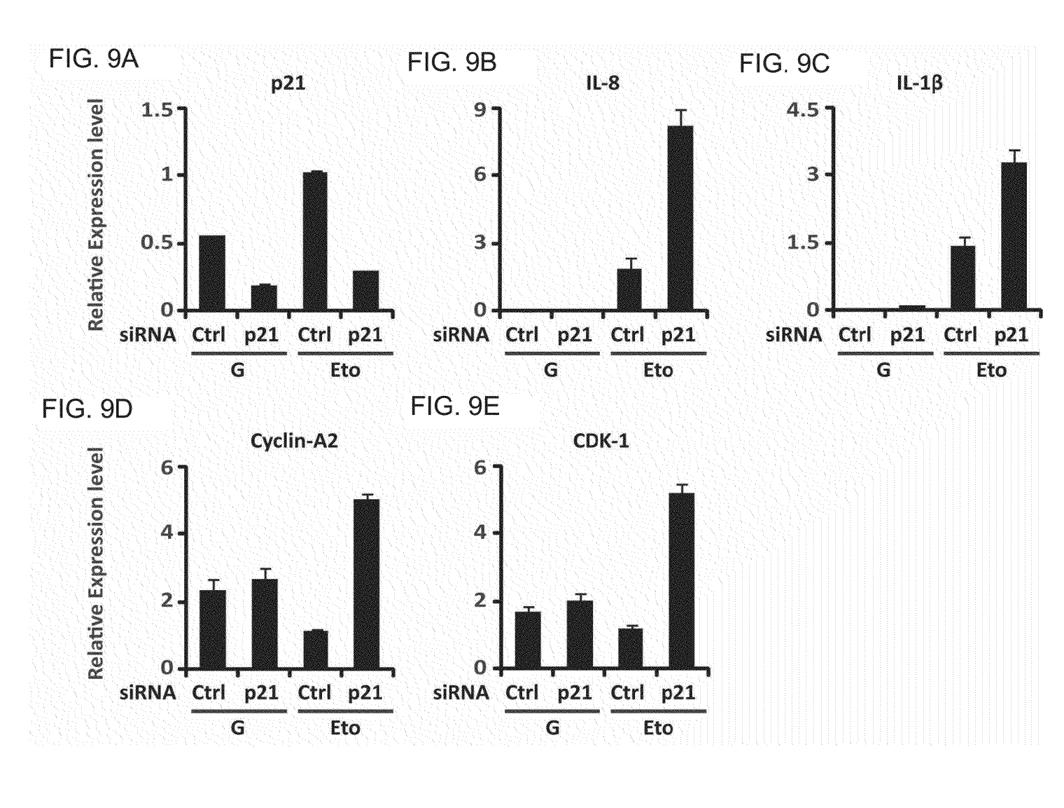

Significant increases in mRNA levels of genes associated with E2F mediated regulation of cell cycle (e.g. Cyclin-A2 and CDK-1) in response to p21 knockdown were noted, as would be expected from the function of p21 as inhibitor of RB protein phosphorylation (FIG. 9). In addition, unexpectedly, it was found that p21 knockdown led to increase in IL-8 and IL-1β mRNA levels, pointing towards an inflammatory response linked to senescent cell death. Therefore, the present inventors conclude that p21 knockdown induces a pro-inflammatory response and cell death in senescent cells. This may lead to an increase in its therapeutic potential because the inflammatory cytokines will recruit the immune system to kill the cells that were not eliminated by the knockdown itself.

Thus, the present inventors propose that combination of direct induction of apoptosis in senescent cells by agents which downregulate Bcl-xL and Bcl-w (which lead to induction of cell death) accompanied by pro-inflammatory response induced by p21 knockdown, should culminate in effective elimination of senescent cells from premalignant lesions, damaged and aged tissues. This will provide important therapeutic impact on the variety of conditions where senescent cells are present.

Thus, according to one aspect of the present invention there is provided a method of treating an inflammatory or fibrotic disease in a subject in need thereof comprising administering to the subject a therapeutically effective amount of an agent which down-regulates an activity and/or an amount of Bcl-xL and/or Bcl-w and/or p21. The term “Bcl-xL” refers to the human protein also known as B-cell lymphoma-extra large, having a sequence as set forth in SEQ ID NO: 21 and homologs and orthologs thereof. The cDNA sequence of human Bcl-xL is set forth in SEQ ID NO: 22.

The term “Bcl-w” refers to the human protein also known as Bcl-2-like protein 2, having a sequence as set forth in SEQ ID NO: 23 and homologs and orthologs thereof. The cDNA sequence of human Bcl-w is set forth in SEQ ID NO: 24.

The term “p21” also known as “cyclin-dependent kinase inhibitor 1” refers to the human protein having a sequence as set forth in SEQ ID NO: 25 and homologs and orthologs thereof. The cDNA sequence of human p21 is set forth in SEQ ID NO: 26.

According to a particular embodiment, the method comprises down-regulation of Bcl-xL and Bcl-w.

According to another embodiment, the method comprises down-regulation of each of Bcl-xL, Bcl-w and p21.

According to still another embodiment, the method comprises down-regulation of p-21 and down-regulation of Bcl-xL.

According to still another embodiment, the method comprises down-regulation of p-21 and down-regulation of Bcl-w.

As used herein, the phrase “downregulating an activity and/or amount” of a target protein refers to a downregulation of at least 10%, at least 20%, at least 30%, at least 40%, at least 50% at least 60%, at least 70%, at least 80% or even at least 90%. In addition, the term “downregulating” may also refer to full inhibition.

Downregulation of Bcl-xL and/or Bcl-w and/or p21 can be effected using chemical agents. Chemical agents known to decrease the activity of Bcl-xL and/or Bcl-w include ABT-737, ABT-263, Gossypol, AT-101, TW-37 and Obatoclax.

According to a particular embodiment, the agent is ABT-737 or ABT-263.

ABT-737 and ABT-263 (ABT-263 being a bioavailable form called “Novatoclax”, Abbot) are currently in Phase II for multiple myeloma, lymphoma, acute leukemia, CLL, small cell lung cancer.

Gossypol (natural) Phase II/III for head and neck tumors, pancreatic cancer. AT-101 (Gossypol derivative; Ascenta Therapeutics) Phase II/III for pancreatic cancer, head and neck cancer, glioma.

TW-37 (Uni Michigan) Phase II for pancreatic cancer, lymphoma.

Obatoclax (GX15-070MS; Gemin X, later Cephalon, now Teva) Phase II for myeloma, myelofibrosis and mantle cell lymphoma.

An example of a chemical agent which down-regulates activity of p21 is disclosed in U.S. Patent Application No. 20110301192, incorporated herein by reference.

Downregulation of Bcl-xL and/or Bcl-w and/or p21 can also be effected on the genomic and/or the transcript level using a variety of molecules which interfere with transcription and/or translation (e.g., RNA silencing agents, Ribozyme, DNAzyme and antisense), or on the protein level using e.g., antagonists, enzymes that cleave the polypeptide and the like.

Following is a list of agents capable of downregulating expression level and/or activity of Bcl-xL and/or Bcl-w and/or p21.

One example, of an agent capable of downregulating Bcl-xL and/or Bcl-w and/or p21 is an antibody or antibody fragment capable of specifically binding thereto. Preferably, the antibody is capable of being internalized by the cell.

The term “antibody” as used in this invention includes intact molecules as well as functional fragments thereof, such as Fab, F(ab′)2, and Fv that are capable of binding to macrophages. These functional antibody fragments are defined as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)2, the fragment of the antibody that can be obtained by treating whole antibody with the enzyme pepsin without subsequent reduction; F(ab′)2 is a dimer of two Fab′ fragments held together by two disulfide bonds; (4) Fv, defined as a genetically engineered fragment containing the variable region of the light chain and the variable region of the heavy chain expressed as two chains; and (5) Single chain antibody (“SCA”), a genetically engineered molecule containing the variable region of the light chain and the variable region of the heavy chain, linked by a suitable polypeptide linker as a genetically fused single chain molecule.

Downregulation of Bcl-xL and/or Bcl-w and/or p21 can be also achieved by RNA silencing. As used herein, the phrase “RNA silencing” refers to a group of regulatory mechanisms [e.g. RNA interference (RNAi), transcriptional gene silencing (TGS), post-transcriptional gene silencing (PTGS), quelling, co-suppression, and translational repression] mediated by RNA molecules which result in the inhibition or “silencing” of the expression of a corresponding protein-coding gene. RNA silencing has been observed in many types of organisms, including plants, animals, and fungi.

As used herein, the term “RNA silencing agent” refers to an RNA which is capable of inhibiting or “silencing” the expression of a target gene. In certain embodiments, the RNA silencing agent is capable of preventing complete processing (e.g, the full translation and/or expression) of an mRNA molecule through a post-transcriptional silencing mechanism. RNA silencing agents include noncoding RNA molecules, for example RNA duplexes comprising paired strands, as well as precursor RNAs from which such small non-coding RNAs can be generated. Exemplary RNA silencing agents include dsRNAs such as siRNAs, miRNAs and shRNAs. In one embodiment, the RNA silencing agent is capable of inducing RNA interference. In another embodiment, the RNA silencing agent is capable of mediating translational repression.

RNA interference refers to the process of sequence-specific post-transcriptional gene silencing in animals mediated by short interfering RNAs (siRNAs). The corresponding process in plants is commonly referred to as post-transcriptional gene silencing or RNA silencing and is also referred to as quelling in fungi. The process of post-transcriptional gene silencing is thought to be an evolutionarily-conserved cellular defense mechanism used to prevent the expression of foreign genes and is commonly shared by diverse flora and phyla. Such protection from foreign gene expression may have evolved in response to the production of double-stranded RNAs (dsRNAs) derived from viral infection or from the random integration of transposon elements into a host genome via a cellular response that specifically destroys homologous single-stranded RNA or viral genomic RNA.

The presence of long dsRNAs in cells stimulates the activity of a ribonuclease III enzyme referred to as dicer. Dicer is involved in the processing of the dsRNA into short pieces of dsRNA known as short interfering RNAs (siRNAs). Short interfering RNAs derived from dicer activity are typically about 21 to about 23 nucleotides in length and comprise about 19 base pair duplexes. The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex.

Accordingly, the present invention contemplates use of dsRNA to downregulate protein expression from mRNA.

According to one embodiment, the dsRNA is greater than 30 bp. The use of long dsRNAs (i.e. dsRNA greater than 30 bp) has been very limited owing to the belief that these longer regions of double stranded RNA will result in the induction of the interferon and PKR response. However, the use of long dsRNAs can provide numerous advantages in that the cell can select the optimal silencing sequence alleviating the need to test numerous siRNAs; long dsRNAs will allow for silencing libraries to have less complexity than would be necessary for siRNAs; and, perhaps most importantly, long dsRNA could prevent viral escape mutations when used as therapeutics.

Various studies demonstrate that long dsRNAs can be used to silence gene expression without inducing the stress response or causing significant off-target effects—see for example [Strat et al., Nucleic Acids Research, 2006, Vol. 34, No. 13 3803-3810; Bhargava A et al. Brain Res. Protoc. 2004; 13:115-125; Diallo M., et al., Oligonucleotides. 2003; 13:381-392; Paddison P. J., et al., Proc. Natl Acad. Sci. USA. 2002; 99:1443-1448; Tran N., et al., FEBS Lett. 2004; 573:127-134].

In particular, the present invention also contemplates introduction of long dsRNA (over 30 base transcripts) for gene silencing in cells where the interferon pathway is not activated (e.g. embryonic cells and oocytes) see for example Billy et al., PNAS 2001, Vol 98, pages 14428-14433 and Diallo et al, Oligonucleotides, Oct. 1, 2003, 13(5): 381-392. doi:10.1089/154545703322617069.

The present invention also contemplates introduction of long dsRNA specifically designed not to induce the interferon and PKR pathways for down-regulating gene expression. For example, Shinagwa and Ishii [Genes & Dev. 17 (11): 1340-1345, 2003] have developed a vector, named pDECAP, to express long double-strand RNA from an RNA polymerase II (Pol II) promoter. Because the transcripts from pDECAP lack both the 5′-cap structure and the 3′-poly(A) tail that facilitate ds-RNA export to the cytoplasm, long ds-RNA from pDECAP does not induce the interferon response.

Another method of evading the interferon and PKR pathways in mammalian systems is by introduction of small inhibitory RNAs (siRNAs) either via transfection or endogenous expression.

The term “siRNA” refers to small inhibitory RNA duplexes (generally between 18-30 basepairs) that induce the RNA interference (RNAi) pathway. Typically, siRNAs are chemically synthesized as 21mers with a central 19 bp duplex region and symmetric 2-base 3′-overhangs on the termini, although it has been recently described that chemically synthesized RNA duplexes of 25-30 base length can have as much as a 100-fold increase in potency compared with 21mers at the same location. The observed increased potency obtained using longer RNAs in triggering RNAi is theorized to result from providing Dicer with a substrate (27mer) instead of a product (21mer) and that this improves the rate or efficiency of entry of the siRNA duplex into RISC.

It has been found that position of the 3′-overhang influences potency of a siRNA and asymmetric duplexes having a 3′-overhang on the antisense strand are generally more potent than those with the 3′-overhang on the sense strand (Rose et al., 2005). This can be attributed to asymmetrical strand loading into RISC, as the opposite efficacy patterns are observed when targeting the antisense transcript.

It will be appreciated that more than one siRNA agent may be used to target Bcl-xL or Bcl-w and/or p21. Thus, the present invention contemplates use of at least two siRNAs that target Bcl-xL, at least three siRNAs that target Bcl-xL, or even at least four siRNAs that target Bcl-xL, each targeting a different sequence in the Bcl-xL gene. Further, the present invention contemplates use of at least two siRNAs that target Bcl-w, at least three siRNAs that target Bcl-w, or even at least four siRNAs that target Bcl-w, each targeting a different sequence in the Bcl-w gene. Further, the present invention contemplates use of at least two siRNAs that target p21, at least three siRNAs that target p21, or even at least four siRNAs that target p21, each targeting a different sequence in the p21 gene.

The strands of a double-stranded interfering RNA (e.g., a siRNA) may be connected to form a hairpin or stem-loop structure (e.g., a shRNA). Thus, as mentioned the RNA silencing agent of the present invention may also be a short hairpin RNA (shRNA).

The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region. The number of nucleotides in the loop is a number between and including 3 to 23, or 5 to 15, or 7 to 13, or 4 to 9, or 9 to 11. Some of the nucleotides in the loop can be involved in base-pair interactions with other nucleotides in the loop. Examples of oligonucleotide sequences that can be used to form the loop include 5′-UUCAAGAGA-3′ (SEQ ID NO: 27; Brummelkamp, T. R. et al. (2002) Science 296: 550) and 5′-UUUGUGUAG-3′ (SEQ ID NO: 28; Castanotto, D. et al. (2002) RNA 8:1454). It will be recognized by one of skill in the art that the resulting single chain oligonucleotide forms a stem-loop or hairpin structure comprising a double-stranded region capable of interacting with the RNAi machinery.

According to another embodiment the RNA silencing agent may be a miRNA. miRNAs are small RNAs made from genes encoding primary transcripts of various sizes. They have been identified in both animals and plants. The primary transcript (termed the “pri-miRNA”) is processed through various nucleolytic steps to a shorter precursor miRNA, or “pre-miRNA.” The pre-miRNA is present in a folded form so that the final (mature) miRNA is present in a duplex, the two strands being referred to as the miRNA (the strand that will eventually basepair with the target) The pre-miRNA is a substrate for a form of dicer that removes the miRNA duplex from the precursor, after which, similarly to siRNAs, the duplex can be taken into the RISC complex. It has been demonstrated that miRNAs can be transgenically expressed and be effective through expression of a precursor form, rather than the entire primary form (Parizotto et al. (2004) Genes & Development 18:2237-2242 and Guo et al. (2005) Plant Cell 17:1376-1386).

Unlike, siRNAs, miRNAs bind to transcript sequences with only partial complementarity (Zeng et al., 2002, Molec. Cell 9:1327-1333) and repress translation without affecting steady-state RNA levels (Lee et al., 1993, Cell 75:843-854; Wightman et al., 1993, Cell 75:855-862). Both miRNAs and siRNAs are processed by Dicer and associate with components of the RNA-induced silencing complex (Hutvagner et al., 2001, Science 293:834-838; Grishok et al., 2001, Cell 106: 23-34; Ketting et al., 2001, Genes Dev. 15:2654-2659; Williams et al., 2002, Proc. Natl. Acad. Sci. USA 99:6889-6894; Hammond et al., 2001, Science 293:1146-1150; Mourlatos et al., 2002, Genes Dev. 16:720-728). A recent report (Hutvagner et al., 2002, Sciencexpress 297:2056-2060) hypothesizes that gene regulation through the miRNA pathway versus the siRNA pathway is determined solely by the degree of complementarity to the target transcript. It is speculated that siRNAs with only partial identity to the mRNA target will function in translational repression, similar to a miRNA, rather than triggering RNA degradation.

Synthesis of RNA silencing agents suitable for use with the present invention can be effected as follows. First, the Bcl-xL and/or Bcl-w mRNA and/or p21 sequence is scanned downstream of the AUG start codon for AA dinucleotide sequences. Occurrence of each AA and the 3′ adjacent 19 nucleotides is recorded as potential siRNA target sites. Preferably, siRNA target sites are selected from the open reading frame, as untranslated regions (UTRs) are richer in regulatory protein binding sites. UTR-binding proteins and/or translation initiation complexes may interfere with binding of the siRNA endonuclease complex [Tuschl ChemBiochem. 2:239-245]. It will be appreciated though, that siRNAs directed at untranslated regions may also be effective, as demonstrated for GAPDH wherein siRNA directed at the 5′ UTR mediated about 90% decrease in cellular GAPDH mRNA and completely abolished protein level. Second, potential target sites are compared to an appropriate genomic database (e.g., human, mouse, rat etc.) using any sequence alignment software, such as the BLAST software available from the NCBI server (wwwdotncbidotnlmdotnihdotgov/BLAST/). Putative target sites which exhibit significant homology to other coding sequences are filtered out.

Qualifying target sequences are selected as template for siRNA synthesis. Preferred sequences are those including low G/C content as these have proven to be more effective in mediating gene silencing as compared to those with G/C content higher than 55%. Several target sites are preferably selected along the length of the target gene for evaluation. For better evaluation of the selected siRNAs, a negative control is preferably used in conjunction. Negative control siRNA preferably include the same nucleotide composition as the siRNAs but lack significant homology to the genome. Thus, a scrambled nucleotide sequence of the siRNA is preferably used, provided it does not display any significant homology to any other gene.

For example, a suitable siRNA capable of downregulating Bcl-xL can be the siRNA of SEQ ID NO: 29, 30 or 31. A suitable siRNA capable of downregulating Bcl-w can be the siRNA of SEQ ID NO: 32, 33 or 34. A suitable siRNA capable of downregulating p21 can be the siRNA of SEQ ID NO: 35, 36 or 37.

It will be appreciated that the RNA silencing agent of the present invention need not be limited to those molecules containing only RNA, but further encompasses chemically-modified nucleotides and non-nucleotides.

In some embodiments, the RNA silencing agent provided herein can be functionally associated with a cell-penetrating peptide.” As used herein, a “cell-penetrating peptide” is a peptide that comprises a short (about 12-30 residues) amino acid sequence or functional motif that confers the energy-independent (i.e., non-endocytotic) translocation properties associated with transport of the membrane-permeable complex across the plasma and/or nuclear membranes of a cell. The cell-penetrating peptide used in the membrane-permeable complex of the present invention preferably comprises at least one non-functional cysteine residue, which is either free or derivatized to form a disulfide link with a double-stranded ribonucleic acid that has been modified for such linkage. Representative amino acid motifs conferring such properties are listed in U.S. Pat. No. 6,348,185, the contents of which are expressly incorporated herein by reference. The cell-penetrating peptides of the present invention preferably include, but are not limited to, penetratin, transportan, pIsl, TAT(48-60), pVEC, MTS, and MAP.

Another agent capable of downregulating Bcl-xL or Bcl-w or p21 is a DNAzyme molecule capable of specifically cleaving an mRNA transcript or DNA sequence thereof. DNAzymes are single-stranded polynucleotides which are capable of cleaving both single and double stranded target sequences (Breaker, R. R. and Joyce, G. Chemistry and Biology 1995; 2:655; Santoro, S. W. & Joyce, G. F. Proc. Natl, Acad. Sci. USA 1997; 943:4262) A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine:pyrimidine junctions (Santoro, S. W. & Joyce, G. F. Proc. Natl, Acad. Sci. USA 199; for rev of DNAzymes see Khachigian, L M [Curr Opin Mol Ther 4:119-21 (2002)].

Examples of construction and amplification of synthetic, engineered DNAzymes recognizing single and double-stranded target cleavage sites have been disclosed in U.S. Pat. No. 6,326,174 to Joyce et al. DNAzymes of similar design directed against the human Urokinase receptor were recently observed to inhibit Urokinase receptor expression, and successfully inhibit colon cancer cell metastasis (Itoh et al, 20002, Abstract 409, Ann Meeting Am Soc Gen Ther wwwdotasgtdotorg). In another application, DNAzymes complementary to bcr-abl oncogenes were successful in inhibiting the oncogenes expression in leukemia cells, and lessening relapse rates in autologous bone marrow transplant in cases of CML and ALL.

Downregulation of Bcl-xL or Bcl-w or p21 can also be effected by using an antisense polynucleotide capable of specifically hybridizing with an mRNA transcript encoding Bcl-xL or Bcl-w.

Design of antisense molecules which can be used to efficiently downregulate Bcl-xL or Bcl-w or p21 must be effected while considering two aspects important to the antisense approach. The first aspect is delivery of the oligonucleotide into the cytoplasm of the appropriate cells, while the second aspect is design of an oligonucleotide which specifically binds the designated mRNA within cells in a way which inhibits translation thereof.

The prior art teaches of a number of delivery strategies which can be used to efficiently deliver oligonucleotides into a wide variety of cell types [see, for example, Luft J Mol Med 76: 75-6 (1998); Kronenwett et al. Blood 91: 852-62 (1998); Rajur et al. Bioconjug Chem 8: 935-40 (1997); Lavigne et al. Biochem Biophys Res Commun 237: 566-71 (1997) and Aoki et al. (1997) Biochem Biophys Res Commun 231: 540-5 (1997)].

In addition, algorithms for identifying those sequences with the highest predicted binding affinity for their target mRNA based on a thermodynamic cycle that accounts for the energetics of structural alterations in both the target mRNA and the oligonucleotide are also available [see, for example, Walton et al. Biotechnol Bioeng 65: 1-9 (1999)].

Such algorithms have been successfully used to implement an antisense approach in cells. For example, the algorithm developed by Walton et al. enabled scientists to successfully design antisense oligonucleotides for rabbit beta-globin (RBG) and mouse tumor necrosis factor-alpha (TNF alpha) transcripts. The same research group has more recently reported that the antisense activity of rationally selected oligonucleotides against three model target mRNAs (human lactate dehydrogenase A and B and rat gp130) in cell culture as evaluated by a kinetic PCR technique proved effective in almost all cases, including tests against three different targets in two cell types with phosphodiester and phosphorothioate oligonucleotide chemistries.

In addition, several approaches for designing and predicting efficiency of specific oligonucleotides using an in vitro system were also published (Matveeva et al., Nature Biotechnology 16: 1374-1375 (1998)].

Another agent capable of downregulating Bcl-xL or Bcl-w or p21 is a ribozyme molecule capable of specifically cleaving an mRNA transcript encoding Bcl-xL or Bcl-w or p21. Ribozymes are being increasingly used for the sequence-specific inhibition of gene expression by the cleavage of mRNAs encoding proteins of interest [Welch et al., Curr Opin Biotechnol. 9:486-96 (1998)]. The possibility of designing ribozymes to cleave any specific target RNA has rendered them valuable tools in both basic research and therapeutic applications. In the therapeutics area, ribozymes have been exploited to target viral RNAs in infectious diseases, dominant oncogenes in cancers and specific somatic mutations in genetic disorders [Welch et al., Clin Diagn Virol. 10:163-71 (1998)]. Most notably, several ribozyme gene therapy protocols for HIV patients are already in Phase 1 trials. More recently, ribozymes have been used for transgenic animal research, gene target validation and pathway elucidation. Several ribozymes are in various stages of clinical trials. ANGIOZYME was the first chemically synthesized ribozyme to be studied in human clinical trials. ANGIOZYME specifically inhibits formation of the VEGF-r (Vascular Endothelial Growth Factor receptor), a key component in the angiogenesis pathway. Ribozyme Pharmaceuticals, Inc., as well as other firms has demonstrated the importance of anti-angiogenesis therapeutics in animal models. HEPTAZYME, a ribozyme designed to selectively destroy Hepatitis C Virus (HCV) RNA, was found effective in decreasing Hepatitis C viral RNA in cell culture assays (Ribozyme Pharmaceuticals, Incorporated—WEB home page).

An additional method of regulating the expression of Bcl-xL or Bcl-w or p21 genes in cells is via triplex forming oligonucleotides (TFOs). Recent studies have shown that TFOs can be designed which can recognize and bind to polypurine/polypirimidine regions in double-stranded helical DNA in a sequence-specific manner. These recognition rules are outlined by Maher III, L. J., et al., Science, 1989; 245:725-730; Moser, H. E., et al., Science, 1987; 238:645-630; Beal, P. A., et al, Science, 1992; 251:1360-1363; Cooney, M., et al., Science, 1988; 241:456-459; and Hogan, M. E., et al., EP Publication 375408. Modification of the oligonucleotides, such as the introduction of intercalators and backbone substitutions, and optimization of binding conditions (pH and cation concentration) have aided in overcoming inherent obstacles to TFO activity such as charge repulsion and instability, and it was recently shown that synthetic oligonucleotides can be targeted to specific sequences (for a recent review see Seidman and Glazer, J Clin Invest 2003; 112:487-94).

In general, the triplex-forming oligonucleotide has the sequence correspondence:

| | | | | |
|---|---|---|---|---|
| oligo | 3′--A | G | G | T |
| duplex | 5′--A | G | C | T |
| duplex | 3′--T | C | G | A |

However, it has been shown that the A-AT and G-GC triplets have the greatest triple helical stability (Reither and Jeltsch, BMC Biochem, 2002, Sep. 12, Epub). The same authors have demonstrated that TFOs designed according to the A-AT and G-GC rule do not form non-specific triplexes, indicating that the triplex formation is indeed sequence specific.

Thus for any given sequence of Bcl-xL or Bcl-w or p21 regulatory region, a triplex forming sequence may be devised. Triplex-forming oligonucleotides preferably are at least 15, more preferably 25, still more preferably 30 or more nucleotides in length, up to 50 or 100 bp.

Transfection of cells (for example, via cationic liposomes) with TFOs, and formation of the triple helical structure with the target DNA induces steric and functional changes, blocking transcription initiation and elongation, allowing the introduction of desired sequence changes in the endogenous DNA and resulting in the specific downregulation of gene expression. Examples of such suppression of gene expression in cells treated with TFOs include knockout of episomal supFG1 and endogenous HPRT genes in mammalian cells (Vasquez et al., Nucl Acids Res. 1999; 27:1176-81, and Puri, et al, J Biol Chem, 2001; 276:28991-98), and the sequence- and target specific downregulation of expression of the Ets2 transcription factor, important in prostate cancer etiology (Carbone, et al, Nucl Acid Res. 2003; 31:833-43), and the pro-inflammatory ICAM-1 gene (Besch et al, J Biol Chem, 2002; 277:32473-79). In addition, Vuyisich and Beal have recently shown that sequence specific TFOs can bind to dsRNA, inhibiting activity of dsRNA-dependent enzymes such as RNA-dependent kinases (Vuyisich and Beal, Nuc. Acids Res 2000; 28:2369-74).

Additionally, TFOs designed according to the abovementioned principles can induce directed mutagenesis capable of effecting DNA repair, thus providing both downregulation and upregulation of expression of endogenous genes (Seidman and Glazer, J Clin Invest 2003; 112:487-94). Detailed description of the design, synthesis and administration of effective TFOs can be found in U.S. Patent Application Nos. 2003 017068 and 2003 0096980 to Froehler et al, and 2002 0128218 and 2002 0123476 to Emanuele et al, and U.S. Pat. No. 5,721,138 to Lawn.

Polynucleotide agents for down-regulating an amount or activity of Bcl-xL and/or Bcl-w and/or p21 are typically administered as part of an expression construct. In this case, the polynucleotide agent is ligated in a nucleic acid construct under the control of a cis-acting regulatory element (e.g. promoter) capable of directing an expression of the agent capable of downregulating Bcl-xL and/or Bcl-w and/or p21 in a constitutive or inducible manner.

The nucleic acid agent may be delivered using an appropriate gene delivery vehicle/method (transfection, transduction, etc.). Optionally an appropriate expression system is used. Examples of suitable constructs include, but are not limited to, pcDNA3, pcDNA3.1 (+/−), pGL3, PzeoSV2 (+/−), pDisplay, pEF/myc/cyto, pCMV/myc/cyto each of which is commercially available from Invitrogen Co. (wwwdotinvitrogendotcom).

The expression construct may also be a virus. Examples of viral constructs include but are not limited to adenoviral vectors, retroviral vectors, vaccinia viral vectors, adeno-associated viral vectors, polyoma viral vectors, alphaviral vectors, rhabdoviral vectors, lenti viral vectors and herpesviral vectors.

A viral construct such as a retroviral construct includes at least one transcriptional promoter/enhancer or locus-defining element(s), or other elements that control gene expression by other means such as alternate splicing, nuclear RNA export, or post-transcriptional modification of messenger. Such vector constructs also include a packaging signal, long terminal repeats (LTRs) or portions thereof, and positive and negative strand primer binding sites appropriate to the virus used, unless it is already present in the viral construct. In addition, such a construct typically includes a signal sequence for secretion of the peptide from a host cell in which it is placed. Preferably, the signal sequence for this purpose is a mammalian signal sequence or the signal sequence of the peptide variants of the present invention. Optionally, the construct may also include a signal that directs polyadenylation, as well as one or more restriction site and a translation termination sequence. By way of example, such constructs will typically include a 5′ LTR, a tRNA binding site, a packaging signal, an origin of second-strand DNA synthesis, and a 3′ LTR or a portion thereof.

Preferably the viral dose for infection is at least 10³, 10⁴, 10⁵, 10⁶, 10⁷, 10⁸, 10⁹, 10¹⁰, 10¹¹, 10¹², 10¹³, 10¹⁴, 10¹⁵ or higher pfu or viral particles.

Double stranded RNA may be synthesized by adding two opposing promoters to the ends of the gene segments, wherein one promoter is placed immediately 5′ to the gene and the opposing promoter is placed immediately 3′ to the gene segment. The dsRNA may then be transcribed with the appropriate polymerase.

The application of small polynucleotide agents (e.g. siRNAs) as potential therapeutic agents requires delivery approaches that will enhance their pharmacological properties. These delivery approaches aim to: (1) increase the retention time of the small polynucleotide agents in the circulatory system by reducing the rate of renal clearance; (2) protect the small polynucleotide agents from serum nucleases; (3) ensure effective biodistribution; (4) facilitate targeting to and uptake of the small polynucleotide agents into the target cells; and (5) promote trafficking to the cytoplasm and uptake into RISC. A variety of approaches have been developed that promote small polynucleotide agent delivery in vivo, including cationic nanoparticles, lipids and liposomes, antibody (Ab)-fusion molecules [Ab-protamine and Ab-poly-arginine, as well as cholesterol and aptamer-conjugated agents. On their own, small polynucleotide agents such as siRNAs fall below the size threshold for renal filtration and are rapidly cleared from the circulatory system. Complexes of small polynucleotide agents and the various delivery reagents remain in the circulation for longer, either because they exceed the size cut-off for renal clearance or because the delivery agents promote association with serum proteins (e.g. serum albumin). In addition, the encapsidation of the small polynucleotide agents into nanoparticles (using either lipid- or cationic-polymer-based systems) helps to shield them from serum nucleases. Ab-fusion molecules have been used to effectively deliver naked, unmodified small polynucleotide agents to specific cell types following intravenous injection. Although the siRNAs are thought to be exposed on the surface of these recombinant Ab-fusion molecules, they were effectively delivered to the target cells, suggesting that complexation with these molecules provides some protection from nucleolytic degradation. The incorporation of chemical modifications to the phosphate backbone, the sugar moiety and the nucleoside bases of the small polynucleotide agents increases its resistance to degradation by serum nucleases. As some of these modifications are detrimental to the silencing efficacy, however, a balance must be maintained between the incorporation of chemical modifications and the inhibitory activity of the small polynucleotide agents. An attractive strategy for decreasing the dosage of the small polynucleotide agents needed to achieve effective silencing and minimizing off-target silencing in bystander cells is the use of delivery agents that target the small polynucleotide agents to specific cell types and tissues. This has been achieved using Abs or ligands that are fused to highly positively charged peptides or proteins, with which the small polynucleotide agents can associate by electrostatic interactions, or by directly conjugating aptamers or ligands to the small polynucleotide agents. These reagents (Abs, ligands and aptamers) can bind with high affinity to cell-surface molecules and deliver the small polynucleotide agents specifically to cells expressing these markers. By combining these targeting reagents with nanoparticles (e.g. immunoliposomes containing lipid nanoparticles coated with specific Abs), the quantity of small polynucleotide agents delivered and, as a consequence, the efficacy of silencing can be increased.

Accordingly, the present invention contemplates use of lipid-based systems for the delivery of these agents. Useful lipids for lipid-mediated transfer of the gene are, for example, DOTMA, DOPE, and DC-Chol [Tonkinson et al., Cancer Investigation, 14(1): 54-65 (1996)]. Recently, it has been shown that Chitosan can be used to deliver nucleic acids to the intestine cells (Chen J. (2004) World J Gastroenterol 10(1):112-116). Other non-lipid based vectors that can be used according to this aspect of the present invention include but are not limited to polylysine and dendrimers, carbon nanotubes, nanogels, polymer based particles.

Since the agents described herein were shown to kill senescent cells, the present inventors propose that these agents may be used to treat subjects having diseases associated with cell senescence.

As used herein, the term “subject” refers to a mammalian subject, preferably a human.

A number of diseases and conditions, which involve an inflammatory response can be treated using the methodology described hereinabove. Examples of such diseases and conditions are summarized infra.

Inflammatory Diseases

Include, but are not limited to, chronic inflammatory diseases and acute inflammatory diseases.

Inflammatory Diseases Associated with Hypersensitivity

Examples of hypersensitivity include, but are not limited to, Type I hypersensitivity, Type II hypersensitivity, Type III hypersensitivity, Type IV hypersensitivity, immediate hypersensitivity, antibody mediated hypersensitivity, immune complex mediated hypersensitivity, T lymphocyte mediated hypersensitivity and DTH.

Type I or immediate hypersensitivity, such as asthma.

Type II hypersensitivity include, but are not limited to, rheumatoid diseases, rheumatoid autoimmune diseases, rheumatoid arthritis (Krenn V. et al., Histol Histopathol 2000 July; 15 (3):791), spondylitis, ankylosing spondylitis (Jan Voswinkel et al., Arthritis Res 2001; 3 (3): 189), systemic diseases, systemic autoimmune diseases, systemic lupus erythematosus (Erikson J. et al., Immunol Res 1998; 17 (1-2):49), sclerosis, systemic sclerosis (Renaudineau Y. et al., Clin Diagn Lab Immunol. 1999 March; 6 (2):156); Chan O T. et al., Immunol Rev 1999 June; 169:107), glandular diseases, glandular autoimmune diseases, pancreatic autoimmune diseases, diabetes, Type I diabetes (Zimmet P. Diabetes Res Clin Pract 1996 October; 34 Suppl:S125), thyroid diseases, autoimmune thyroid diseases, Graves' disease (Orgiazzi J. Endocrinol Metab Clin North Am 2000 June; 29 (2):339), thyroiditis, spontaneous autoimmune thyroiditis (Braley-Mullen H. and Yu S, J Immunol 2000 Dec. 15; 165 (12):7262), Hashimoto's thyroiditis (Toyoda N. et al., Nippon Rinsho 1999 August; 57 (8):1810), myxedema, idiopathic myxedema (Mitsuma T. Nippon Rinsho. 1999 August; 57 (8):1759); autoimmune reproductive diseases, ovarian diseases, ovarian autoimmunity (Garza K M. et al., J Reprod Immunol 1998 February; 37 (2):87), autoimmune anti-sperm infertility (Diekman A B. et al., Am J Reprod Immunol. 2000 March; 43 (3):134), repeated fetal loss (Tincani A. et al., Lupus 1998; 7 Suppl 2:S107-9), neurodegenerative diseases, neurological diseases, neurological autoimmune diseases, multiple sclerosis (Cross A H. et al., J Neuroimmunol 2001 Jan. 1; 112 (1-2):1), Alzheimer's disease (Oron L. et al., J Neural Transm Suppl. 1997; 49:77), myasthenia gravis (Infante A J. And Kraig E, Int Rev Immunol 1999; 18 (1-2):83), motor neuropathies (Kornberg A J. J Clin Neurosci. 2000 May; 7 (3):191), Guillain-Barre syndrome, neuropathies and autoimmune neuropathies (Kusunoki S. Am J Med Sci. 2000 April; 319 (4):234), myasthenic diseases, Lambert-Eaton myasthenic syndrome (Takamori M. Am J Med Sci. 2000 April; 319 (4):204), paraneoplastic neurological diseases, cerebellar atrophy, paraneoplastic cerebellar atrophy, non-paraneoplastic stiff man syndrome, cerebellar atrophies, progressive cerebellar atrophies, encephalitis, Rasmussen's encephalitis, amyotrophic lateral sclerosis, Sydeham chorea, Gilles de la Tourette syndrome, polyendocrinopathies, autoimmune polyendocrinopathies (Antoine J C. and Honnorat J. Rev Neurol (Paris) 2000 January; 156 (1):23); neuropathies, dysimmune neuropathies (Nobile-Orazio E. et al., Electroencephalogr Clin Neurophysiol Suppl 1999; 50:419); neuromyotonia, acquired neuromyotonia, arthrogryposis multiplex congenita (Vincent A. et al., Ann N Y Acad Sci. 1998 May 13; 841:482), cardiovascular diseases, cardiovascular autoimmune diseases, atherosclerosis (Matsuura E. et al., Lupus. 1998; 7 Suppl 2:S135), myocardial infarction (Vaarala O. Lupus. 1998; 7 Suppl 2:S132), thrombosis (Tincani A. et al., Lupus 1998; 7 Suppl 2:S107-9), granulomatosis, Wegener's granulomatosis, arteritis, Takayasu's arteritis and Kawasaki syndrome (Praprotnik S. et al., Wien Klin Wochenschr 2000 Aug. 25; 112 (15-16):660); anti-factor VIII autoimmune disease (Lacroix-Desmazes S. et al., Semin Thromb Hemost. 2000; 26 (2):157); vasculitises, necrotizing small vessel vasculitises, microscopic polyangiitis, Churg and Strauss syndrome, glomerulonephritis, pauci-immune focal necrotizing glomerulonephritis, crescentic glomerulonephritis (Noel L H. Ann Med Interne (Paris). 2000 May; 151 (3):178); antiphospholipid syndrome (Flamholz R. et al., J Clin Apheresis 1999; 14 (4):171); heart failure, agonist-like beta-adrenoceptor antibodies in heart failure (Wallukat G. et al., Am J Cardiol. 1999 Jun. 17; 83 (12A):75H), thrombocytopenic purpura (Moccia F. Ann Ital Med Int. 1999 April-June; 14 (2):114); hemolytic anemia, autoimmune hemolytic anemia (Efremov D G. et al., Leuk Lymphoma 1998 January; 28 (3-4):285), gastrointestinal diseases, autoimmune diseases of the gastrointestinal tract, intestinal diseases, chronic inflammatory intestinal disease (Garcia Herola A. et al., Gastroenterol Hepatol. 2000 January; 23 (1):16), celiac disease (Landau Y E. and Shoenfeld Y. Harefuah 2000 Jan. 16; 138 (2):122), autoimmune diseases of the musculature, myositis, autoimmune myositis, Sjogren's syndrome (Feist E. et al., Int Arch Allergy Immunol 2000 September; 123 (1):92); smooth muscle autoimmune disease (Zauli D. et al., Biomed Pharmacother 1999 June; 53 (5-6):234), hepatic diseases, hepatic autoimmune diseases, autoimmune hepatitis (Manns M P. J Hepatol 2000 August; 33 (2):326) and primary biliary cirrhosis (Strassburg C P. et al., Eur J Gastroenterol Hepatol. 1999 June; 11 (6):595).

Type IV or T cell mediated hypersensitivity, include, but are not limited to, rheumatoid diseases, rheumatoid arthritis (Tisch R, McDevitt H O. Proc Natl Acad Sci USA 1994 Jan. 18; 91 (2):437), systemic diseases, systemic autoimmune diseases, systemic lupus erythematosus (Datta S K., Lupus 1998; 7 (9):591), glandular diseases, glandular autoimmune diseases, pancreatic diseases, pancreatic autoimmune diseases, Type 1 diabetes (Castano L. and Eisenbarth G S. Ann. Rev. Immunol. 8:647); thyroid diseases, autoimmune thyroid diseases, Graves' disease (Sakata S. et al., Mol Cell Endocrinol 1993 March; 92 (1):77); ovarian diseases (Garza K M. et al., J Reprod Immunol 1998 February; 37 (2):87), prostatitis, autoimmune prostatitis (Alexander R B. et al., Urology 1997 December; 50 (6):893), polyglandular syndrome, autoimmune polyglandular syndrome, Type I autoimmune polyglandular syndrome (Hara T. et al., Blood. 1991 Mar. 1; 77 (5):1127), neurological diseases, autoimmune neurological diseases, multiple sclerosis, neuritis, optic neuritis (Soderstrom M. et al., J Neurol Neurosurg Psychiatry 1994 May; 57 (5):544), myasthenia gravis (Oshima M. et al., Eur J Immunol 1990 December; 20 (12):2563), stiff-man syndrome (Hiemstra H S. et al., Proc Natl Acad Sci USA 2001 Mar. 27; 98 (7):3988), cardiovascular diseases, cardiac autoimmunity in Chagas' disease (Cunha-Neto E. et al., J Clin Invest 1996 Oct. 15; 98 (8):1709), autoimmune thrombocytopenic purpura (Semple J W. et al., Blood 1996 May 15; 87 (10):4245), anti-helper T lymphocyte autoimmunity (Caporossi A P. et al., Viral Immunol 1998; 11 (1):9), hemolytic anemia (Sallah S. et al., Ann Hematol 1997 March; 74 (3):139), hepatic diseases, hepatic autoimmune diseases, hepatitis, chronic active hepatitis (Franco A. et al., Clin Immunol Immunopathol 1990 March; 54 (3):382), biliary cirrhosis, primary biliary cirrhosis (Jones D E. Clin Sci (Colch) 1996 November; 91 (5):551), nephric diseases, nephric autoimmune diseases, nephritis, interstitial nephritis (Kelly C J. J Am Soc Nephrol 1990 August; 1 (2):140), connective tissue diseases, ear diseases, autoimmune connective tissue diseases, autoimmune ear disease (Yoo T J. et al., Cell Immunol 1994 August; 157 (1):249), disease of the inner ear (Gloddek B. et al., Ann N Y Acad Sci 1997 Dec. 29; 830:266), skin diseases, cutaneous diseases, dermal diseases, bullous skin diseases, pemphigus vulgaris, bullous pemphigoid and pemphigus foliaceus.

Examples of delayed type hypersensitivity include, but are not limited to, contact dermatitis and drug eruption.

Examples of types of T lymphocyte mediating hypersensitivity include, but are not limited to, helper T lymphocytes and cytotoxic T lymphocytes.

Examples of helper T lymphocyte-mediated hypersensitivity include, but are not limited to, Tₕ1 lymphocyte mediated hypersensitivity and Tₕ2 lymphocyte mediated hypersensitivity.

Autoimmune Diseases

Include, but are not limited to, cardiovascular diseases, rheumatoid diseases, glandular diseases, gastrointestinal diseases, cutaneous diseases, hepatic diseases, neurological diseases, muscular diseases, nephric diseases, diseases related to reproduction, connective tissue diseases and systemic diseases.

Examples of autoimmune cardiovascular diseases include, but are not limited to atherosclerosis (Matsuura E. et al., Lupus. 1998; 7 Suppl 2:S135), myocardial infarction (Vaarala O. Lupus. 1998; 7 Suppl 2:S132), thrombosis (Tincani A. et al., Lupus 1998; 7 Suppl 2:S107-9), Wegener's granulomatosis, Takayasu's arteritis, Kawasaki syndrome (Praprotnik S. et al., Wien Klin Wochenschr 2000 Aug. 25; 112 (15-16):660), anti-factor VIII autoimmune disease (Lacroix-Desmazes S. et al., Semin Thromb Hemost. 2000; 26 (2):157), necrotizing small vessel vasculitis, microscopic polyangiitis, Churg and Strauss syndrome, pauci-immune focal necrotizing and crescentic glomerulonephritis (Noel L H. Ann Med Interne (Paris). 2000 May; 151 (3):178), antiphospholipid syndrome (Flamholz R. et al., J Clin Apheresis 1999; 14 (4):171), antibody-induced heart failure (Wallukat G. et al., Am J Cardiol. 1999 Jun. 17; 83 (12A):75H), thrombocytopenic purpura (Moccia F. Ann Ital Med Int. 1999 April-June; 14 (2):114; Semple J W. et al., Blood 1996 May 15; 87 (10):4245), autoimmune hemolytic anemia (Efremov D G. et al., Leuk Lymphoma 1998 January; 28 (3-4):285; Sallah S. et al., Ann Hematol 1997 March; 74 (3):139), cardiac autoimmunity in Chagas' disease (Cunha-Neto E. et al., J Clin Invest 1996 Oct. 15; 98 (8):1709) and anti-helper T lymphocyte autoimmunity (Caporossi A P. et al., Viral Immunol 1998; 11 (1):9).

Examples of autoimmune rheumatoid diseases include, but are not limited to rheumatoid arthritis (Krenn V. et al., Histol Histopathol 2000 July; 15 (3):791; Tisch R, McDevitt H O. Proc Natl Acad Sci units S A 1994 Jan. 18; 91 (2):437) and ankylosing spondylitis (Jan Voswinkel et al., Arthritis Res 2001; 3 (3): 189).

Examples of autoimmune glandular diseases include, but are not limited to, pancreatic disease, Type I diabetes, thyroid disease, Graves' disease, thyroiditis, spontaneous autoimmune thyroiditis, Hashimoto's thyroiditis, idiopathic myxedema, ovarian autoimmunity, autoimmune anti-sperm infertility, autoimmune prostatitis and Type I autoimmune polyglandular syndrome. Diseases include, but are not limited to autoimmune diseases of the pancreas, Type 1 diabetes (Castano L. and Eisenbarth G S. Ann. Rev. Immunol. 8:647; Zimmet P. Diabetes Res Clin Pract 1996 October; 34 Suppl:S125), autoimmune thyroid diseases, Graves' disease (Orgiazzi J. Endocrinol Metab Clin North Am 2000 June; 29 (2):339; Sakata S. et al., Mol Cell Endocrinol 1993 March; 92 (1):77), spontaneous autoimmune thyroiditis (Braley-Mullen H. and Yu S, J Immunol 2000 Dec. 15; 165 (12):7262), Hashimoto's thyroiditis (Toyoda N. et al., Nippon Rinsho 1999 August; 57 (8):1810), idiopathic myxedema (Mitsuma T. Nippon Rinsho. 1999 August; 57 (8):1759), ovarian autoimmunity (Garza K M. et al., J Reprod Immunol 1998 February; 37 (2):87), autoimmune anti-sperm infertility (Diekman A B. et al., Am J Reprod Immunol. 2000 March; 43 (3):134), autoimmune prostatitis (Alexander R B. et al., Urology 1997 December; 50 (6):893) and Type I autoimmune polyglandular syndrome (Hara T. et al., Blood. 1991 Mar. 1; 77 (5):1127).

Examples of autoimmune gastrointestinal diseases include, but are not limited to, chronic inflammatory intestinal diseases (Garcia Herola A. et al., Gastroenterol Hepatol. 2000 January; 23 (1):16), celiac disease (Landau Y E. and Shoenfeld Y. Harefuah 2000 Jan. 16; 138 (2):122), colitis, ileitis and Crohn's disease.

Examples of autoimmune cutaneous diseases include, but are not limited to, autoimmune bullous skin diseases, such as, but are not limited to, pemphigus vulgaris, bullous pemphigoid and pemphigus foliaceus.

Examples of autoimmune hepatic diseases include, but are not limited to, hepatitis, autoimmune chronic active hepatitis (Franco A. et al., Clin Immunol Immunopathol 1990 March; 54 (3):382), primary biliary cirrhosis (Jones D E. Clin Sci (Colch) 1996 November; 91 (5):551; Strassburg C P. et al., Eur J Gastroenterol Hepatol. 1999 June; 11 (6):595) and autoimmune hepatitis (Manns M P. J Hepatol 2000 August; 33 (2):326).

Examples of autoimmune neurological diseases include, but are not limited to, multiple sclerosis (Cross A H. et al., J Neuroimmunol 2001 Jan. 1; 112 (1-2):1), Alzheimer's disease (Oron L. et al., J Neural Transm Suppl. 1997; 49:77), myasthenia gravis (Infante A J. And Kraig E, Int Rev Immunol 1999; 18 (1-2):83; Oshima M. et al., Eur J Immunol 1990 December; 20 (12):2563), neuropathies, motor neuropathies (Kornberg A J. J Clin Neurosci. 2000 May; 7 (3):191); Guillain-Barre syndrome and autoimmune neuropathies (Kusunoki S. Am J Med Sci. 2000 April; 319 (4):234), myasthenia, Lambert-Eaton myasthenic syndrome (Takamori M. Am J Med Sci. 2000 April; 319 (4):204); paraneoplastic neurological diseases, cerebellar atrophy, paraneoplastic cerebellar atrophy and stiff-man syndrome (Hiemstra H S. et al., Proc Natl Acad Sci units S A 2001 Mar. 27; 98 (7):3988); non-paraneoplastic stiff man syndrome, progressive cerebellar atrophies, encephalitis, Rasmussen's encephalitis, amyotrophic lateral sclerosis, Sydeham chorea, Gilles de la Tourette syndrome and autoimmune polyendocrinopathies (Antoine J C. and Honnorat J. Rev Neurol (Paris) 2000 January; 156 (1):23); dysimmune neuropathies (Nobile-Orazio E. et al., Electroencephalogr Clin Neurophysiol Suppl 1999; 50:419); acquired neuromyotonia, arthrogryposis multiplex congenita (Vincent A. et al., Ann N Y Acad Sci. 1998 May 13; 841:482), neuritis, optic neuritis (Soderstrom M. et al., J Neurol Neurosurg Psychiatry 1994 May; 57 (5):544) and neurodegenerative diseases.

Examples of autoimmune muscular diseases include, but are not limited to, myositis, autoimmune myositis and primary Sjogren's syndrome (Feist E. et al., Int Arch Allergy Immunol 2000 September; 123 (1):92) and smooth muscle autoimmune disease (Zauli D. et al., Biomed Pharmacother 1999 June; 53 (5-6):234).

Examples of autoimmune nephric diseases include, but are not limited to, nephritis and autoimmune interstitial nephritis (Kelly C J. J Am Soc Nephrol 1990 August; 1 (2):140).

Examples of autoimmune diseases related to reproduction include, but are not limited to, repeated fetal loss (Tincani A. et al., Lupus 1998; 7 Suppl 2:S107-9).

Examples of autoimmune connective tissue diseases include, but are not limited to, ear diseases, autoimmune ear diseases (Yoo T J. et al., Cell Immunol 1994 August; 157 (1):249) and autoimmune diseases of the inner ear (Gloddek B. et al., Ann N Y Acad Sci 1997 Dec. 29; 830:266).

Examples of autoimmune systemic diseases include, but are not limited to, systemic lupus erythematosus (Erikson J. et al., Immunol Res 1998; 17 (1-2):49) and systemic sclerosis (Renaudineau Y. et al., Clin Diagn Lab Immunol. 1999 March; 6 (2):156); Chan O T. et al., Immunol Rev 1999 June; 169:107).

Infectious Diseases

Examples of infectious diseases include, but are not limited to, chronic infectious diseases, subacute infectious diseases, acute infectious diseases, viral diseases, bacterial diseases, protozoan diseases, parasitic diseases, fungal diseases, mycoplasma diseases and prion diseases.

Graft Rejection Diseases

Examples of diseases associated with transplantation of a graft include, but are not limited to, graft rejection, chronic graft rejection, subacute graft rejection, hyperacute graft rejection, acute graft rejection and graft versus host disease.

Allergic Diseases

Examples of allergic diseases include, but are not limited to, asthma, hives, urticaria, pollen allergy, dust mite allergy, venom allergy, cosmetics allergy, latex allergy, chemical allergy, drug allergy, insect bite allergy, animal dander allergy, stinging plant allergy, poison ivy allergy and food allergy.

According to a particular embodiment, the agents (and combinations thereof) are used to treat pre-malignant lesions.

As used herein, the phrase “pre-malignant lesion” refers to a mass of cells and/or tissue having increased probability of transforming into a malignant tumor. Examples of pre-malignant lesions include, but are not limited to, adenomatous polyps, Barrett's esophagus, IPMN (Intraductal Papillary Mucinus Neoplasia), DCIS (Ductal Carcinoma in Situ) in the breast, leukoplakia and erythroplakia. Thus, the pre-malignant lesion which is treated using the agents of this aspect of the present invention can transform into a malignant solid or non-solid (e.g., hematological malignancies) cancer (or tumor). According to a particular embodiment, the pre-malignant lesion which is treated using the agents of the present invention is an adenomatous polyp of the colon, an adenomatous polyp of the rectum, an adenomatous polyp of the small bowel and Barrett's esophagus.

Examples of fibrotic diseases include diseases of an epithelial barrier tissue, diseases of the skin, lung or gut.

Contemplated fibrotic diseases which may be treated using the agents described herein include but are not limited to eosinophilic esophagitis, hypereosinophilic syndromes (HES), Loeffler's endomyocarditis, endomyocardial fibrosis, idiopathic pulmonary fibrosis, and scleroderma.

According to a particular embodiment the agents are used for treating liver fibrosis, wound healing, skin fibrosis, pulmonary disease, kidney fibrosis, prostatitis, atherosclerosis, arthritis, osteoporosis or pancreatitis.

An exemplary pulmonary disease contemplated by the present invention is chronic obstructive pulmonary disease (COPD).

According to sill another embodiment, the disease is associated with cartilage degeneration—e.g. arthritis.

According to still another embodiment, the disease is associated with bone degeneration—e.g. osteoporosis.

According to still another embodiment, the disease is not cancer.

The agents of the present invention (and combinations thereof) may be provided per se or may be formulated in compositions intended for a particular use. It will be appreciated that combinations of the agents described herein may be provided in a single formulation or may be provided in individual compositions.

Contemplated compositions include those that comprise an agent which downregulates of Bcl-xL and an agent which downregulates Bcl-w (e.g. siRNA agents). Another contemplated composition is one which includes an agent which downregulates of Bcl-xL and an agent which downregulates Bcl-w (e.g. siRNA agents) and an agent which downregulates p21 (e.g. siRNA agent).

Another contemplated composition is one which includes an agent which downregulates of Bcl-xL and Bcl-w (e.g. chemical agent) and an agent which downregulates p21 (e.g. siRNA agent).

Another contemplated composition is one which includes an agent which downregulates of Bcl-xL and Bcl-w (e.g. chemical agent) and an agent which downregulates p21 (e.g. chemical agent).

Further, the present inventors contemplate providing combinations of the agents individually packed in a single article of manufacture.

Thus, one contemplated article of manufacture includes an agent which downregulates of Bcl-xL and an agent which downregulates Bcl-w (e.g. siRNA agents).

Another contemplated article of manufacture is one which includes an agent which downregulates of Bcl-xL and an agent which downregulates Bcl-w (e.g. siRNA agents) and an agent which downregulates p21 (e.g. siRNA agent).

Another contemplated article of manufacture is one which includes an agent which downregulates of Bcl-xL and Bcl-w (e.g. chemical agent) and an agent which downregulates p21 (e.g. siRNA agent).

Another contemplated article of manufacture is one which includes an agent which downregulates of Bcl-xL and Bcl-w (e.g. chemical agent) and an agent which downregulates p21 (e.g. chemical agent).

Since the agents of the present invention selectively kill senescent cells, the present inventors contemplate that another use thereof is in cosmetic compositions as anti-aging agents for rejuvenating the skin. Thus, the agents of the present invention may be formulated for cosmetics.

Such compositions typically comprise pharmaceutically acceptable excipient, notably dermatologically acceptable suitable for external topical application.

The cosmetic composition according to the present invention may further comprise at least one pharmaceutical adjuvant known to the person skilled in the art, selected from thickeners, preservatives, fragrances, colorants, chemical or mineral filters, moisturizing agents, thermal spring water, etc.

The composition may comprise at least one agent selected from a sebum-regulating agent, an antibacterial agent, an antifungal agent, a keratolytic agent, a keratoregulating agent, an astringent, an anti-inflammatory/anti-irritant, an antioxidant/free-radical scavenger, a cicatrizing agent, an anti-aging agent and/or a moisturizing agent.

The term “sebum-regulating agent” refers, for example, to 5-α-reductase inhibitors, notably the active agent 5-α-Avocuta® sold by Laboratories Expanscience. Zinc and gluconate salts thereof, salicylate and pyroglutamic acid, also have sebum-suppressing activity. Mention may also be made of spironolactone, an anti-androgen and aldosterone antagonist, which significantly reduces the sebum secretion rate after 12 weeks of application. Other extracted molecules, for example from seeds of the pumpkin Cucurbita pepo, and squash seed oil, as well as palm cabbage, limit sebum production by inhibiting 5-α-reductase transcription and activity. Other sebum-regulating agents of lipid origin that act on sebum quality, such as linoleic acid, are of interest. The terms “anti-bacterial agent” and “antifungal agent” refer to molecules that limit the growth of or destroy pathogenic microorganisms such as certain bacteria like P. acnes or certain fungi (Malassezia furfur). The most traditional are preservatives generally used in cosmetics or nutraceuticals, molecules with anti-bacterial activity (pseudo-preservatives) such as caprylic derivatives (capryloyl glycine, glyceryl caprylate, etc.), such as hexanediol and sodium levulinate, zinc and copper derivatives (gluconate and PCA), phytosphingosine and derivatives thereof, benzoyl peroxide, piroctone olamine, zinc pyrithione, selenium sulfide, econazole, ketoconazole, or local antibiotics such as erythromycin and clindamycin, etc.

The terms “keratoregulating agent” and “keratolytic agent” refer to an agent that regulates or helps the elimination of dead cells of the stratum corneum of the epidermis. The most commonly used keratoregulating/keratolytic agents include: alpha-hydroxy acids (AHAs) of fruits (citric acid, glycolic acid, malic acid, lactic acid, etc.), AHA esters, combinations of AHAs with other molecules such as the combination of malic acid and almond proteins (Keratolite®), the combination of glycolic acid or lactic acid with arginine or the combination of hydroxy acid with lipid molecules such as LHA® (lipo-hydroxy acid), amphoteric hydroxy acid complexes (AHCare), willow bark (Salix alba bark extract), azelaic acid and salts and esters thereof, salicylic acid and derivatives thereof such as capryloyl salicylic acid or in combination with other molecules such as the combination of salicylic acid and polysaccharide (beta-hydroxy acid, or BHA), tazarotene, adapalene, as well as molecules of the retinoid family such as tretinoin, retinaldehyde, isotretinoin and retinol.

The term “astringent” refers to an agent that helps constrict pores, the most commonly used being polyphenols, zinc derivatives and witch hazel.

The term “anti-inflammatory/anti-irritant” refers to an agent that limits the inflammatory reaction led by cytokines or arachidonic acid metabolism mediators and has soothing and anti-irritating properties. The most traditional are glycyrrhetinic acid (licorice derivative) and salts and esters thereof, alpha-bisabolol, Ginkgo biloba, Calendula, lipoic acid, beta-carotene, vitamin B3 (niacinamide, nicotinamide), vitamin E, vitamin C, vitamin B12, flavonoids (green tea, quercetin, etc.), lycopene or lutein, avocado sugars, avocado oleodistillate, arabinogalactan, lupin peptides, lupin total extract, quinoa peptide extract, Cycloceramide′® (oxazoline derivative), anti-glycation agents such as carnosine, N-acetyl-cysteine, isoflavones such as, for example, genistein/genistin, daidzein/daidzin, spring water or thermal spring water (eau d′Avene, eau de la Roche Posay, eau de Saint Gervais, eau d′Uriage, eau de Gamarde), goji extracts (Lycium barbarum), plant amino acid peptides or complexes, topical dapsone, or anti-inflammatory drugs.

The term “antioxidant” refers to a molecule that decreases or prevents the oxidation of other chemical substances. The antioxidants/free-radical scavengers that may be used in combination are advantageously selected from the group comprised of thiols and phenols, licorice derivatives such as glycyrrhetinic acid and salts and esters thereof, alpha-bisabolol, Ginkgo biloba extract, Calendula extract, Cycloceramide® (oxazoline derivative), avocado peptides, trace elements such as copper, zinc and selenium, lipoic acid, vitamin B12, vitamin B3 (niacinamide, nicotinamide), vitamin C, vitamin E, coenzyme Q10, hill, glutathione, butylated hydroxytoluene (BHT), butylated hydroxyanisole (BHA), lycopene or lutein, beta-carotene, the family of polyphenols such as tannins, phenolic acids, anthocyanins, flavonoids such as, for example, extracts of green tea, of red berries, of cocoa, of grapes, of Passiflora incarnata or of Citrus, or isoflavones such as, for example, genistein/genistin and daidzein/daidzin. The group of antioxidants further includes anti-glycation agents such as carnosine or certain peptides, N-acetyl-cysteine, as well as antioxidant or free-radical scavenging enzymes such as superoxide dismutase (SOD), catalase, glutathione peroxidase, thioredoxin reductase and agonists thereof.

The agents that cicatrize/repair the barrier function which may be used in combination are advantageously vitamin A, panthenol (vitamin B5), Avocadofurane®, avocado sugars, lupeol, maca peptide extract, quinoa peptide extract, arabinogalactan, zinc oxide, magnesium, silicon, madecassic or asiatic acid, dextran sulfate, coenzyme Q10, glucosamine and derivatives thereof, chondroitin sulfate and on the whole glycosaminoglycans (GAGs), dextran sulfate, ceramides, cholesterol, squalane, phospholipids, fermented or unfermented soya peptides, plant peptides, marine, plant or biotechnological polysaccharides such as algae extracts or fern extracts, trace elements, extracts of tannin-rich plants such as tannins derived from gallic acid called gallic or hydrolysable tannins, initially found in oak gall, and catechin tannins resulting from the polymerization of flavan units whose model is provided by the catechu (Acacia catechu). The trace elements that may be used are advantageously selected from the group comprised of copper, magnesium, manganese, chromium, selenium, silicon, zinc and mixtures thereof.

The anti-aging agents that can act in combination to treat acne in mature subjects are antioxidants and in particular vitamin C, vitamin A, retinol, retinal, hyaluronic acid of any molecular weight, Avocadofurane®, lupin peptides and maca peptide extract.

The most commonly used moisturizers/emollients are glycerin or derivatives thereof, urea, pyrrolidone carboxylic acid and derivatives thereof, hyaluronic acid of any molecular weight, glycosaminoglycans and any other polysaccharides of marine, plant or biotechnological origin such as, for example, xanthan gum, Fucogel®, certain fatty acids such as lauric acid, myristic acid, monounsaturated and polyunsaturated omega-3, -6, -7 and -9 fatty acids (linoleic acid, palmitoleic acid, etc.), sunflower oleodistillate, avocado peptides and cupuacu butter.

For treatment of diseases, the agents of the present invention may for formulated in pharmaceutical compositions.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the agents which downregulate Bcl-xL, Bcl-w and/or p21 accountable for the biological effect. It will be appreciated that the pharmaceutical compositions may comprise additional active agents known to be useful in treating a particular disease. Thus, for example for treatment of skin fibrotic diseases, the present inventors contemplate pharmaceutical compositions comprising the above described agents together with at least one sebum-regulating agent, an antibacterial agent, an antifungal agent, a keratolytic agent, a keratoregulating agent, an astringent, an anti-inflammatory/anti-irritant, an antioxidant/free-radical scavenger, a cicatrizing agent, an anti-aging agent and/or a moisturizing agent, as described herein above.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, inrtaperitoneal, intranasal, or intraocular injections.

According to a particular embodiment, the route of administration is via topical delivery.

Alternately, one may administer the pharmaceutical composition in a local rather than systemic manner, for example, via injection of the pharmaceutical composition directly into a tissue region of a patient.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum Arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients (e.g. siRNA agents) effective to prevent, alleviate or ameliorate symptoms of a disorder (e.g., fibrotic or inflammatory disease) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated from animal models (e.g. mouse models of liver fibrosis induced by CCl₄, mouse model of pancreatitis induced by Caerulein, mouse model of COPD) to achieve a desired concentration or titer. Such information can be used to more accurately determine useful doses in humans.

Toxicity and therapeutic efficacy of the active ingredients described herein can be determined by standard pharmaceutical procedures in experimental animals. The data obtained from these animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide cell numbers sufficient to induce normoglycemia (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as if further detailed above.

It is expected that during the life of a patent maturing from this application many relevant agents capable of down-regulating of Bcl-xL and/or Bcl-w and/or p21 will be developed and the scope of the phrase “agents capable of down-regulating” is intended to include all such new technologies a priori.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### General Materials and Methods

Tissue Culture:

Human primary fibroblasts (IMR-90, BJ) were obtained from ATCC MEFs were prepared from day 13.5 embryos. All cultures were maintained in DMEM supplemented with 10% fetal bovine serum (Hyclone). Senescence was induced by treatment with Etoposide (50 mM, Sigma), or the introduction of oncogenic H-ras^{v12} using infections into IMR-90 cells (as described by Narita, 2003).

Immunoblotting:

Cells were lysed in RIPA buffer. Equal amounts of protein were separated on 12% SDS-polyacrylamide gels and transferred to PVDF membranes. Detection was performed using the following antibodies: anti-Rb (9313), anti-cleaved parp (9541), anti-cleaved caspase-3 (9661), anti-phospho-p53 (9284), anti-mouse-p53 (2524), anti-phospho-NF-κB (3033), anti-Bcl-2 (2870), anti-Bcl-w (2724) and anti-Bcl-xL (2764) were purchased from Cell Signaling Technology. Anti-human p53 (DO1 and PAb1801). Anti-p16 (sc-759), anti-mouse-p21 (sc-397), anti-NFκB p65 (sc-372) and anti-α-Tubulin (sc-9104) were obtained from Santa Cruz Biotechnology. Anti-human-p21 (556431) was obtained from BD Pharmingen, Anti-Mcl-1 (1239-1) was obtained from Epitomics.

RNA Isolation and Quantitative RT-PCR:

For quantitative RT-PCR, total RNA was isolated using the NucleoSpin kit (Macherey Nagel, Düren, Germany). A 1 μg aliquot of the total RNA was reverse-transcribed using Moloney murine leukemia virus reverse transcriptase (Promega) and random hexamer primers (Applied Biosystems). Realtime PCR was done using Platinum SYBR Green qPCR SuperMix (Invitrogen) on an ABI StepOnePlus instrument (Applied Biosystems).

The values for specific genes were normalized to GAPDH. Primer sequences were as follows: (1) GAPDH forward, 5′-GACAGTCAGCCGCATCTTC-3′ (SEQ ID NO: 1); reverse, 5′-CGTTGACTCCGACCTTCAC-3′ (SEQ ID NO: 2); (2) Bcl-2 forward, 5′-ACTGGAGAGTGCTGAAGATTGATG-3′ (SEQ ID NO: 3); reverse, 5′-CTACTTCCTCTGTGATGTTGTATTTTTTAAG-3′ (SEQ ID NO: 4); (3) Bcl-w forward, 5′-TGACACCTGGGTGGAAAGAG-3′ (SEQ ID NO: 5); reverse, 5′-CCACTGTGGTCCCATCTAAG-3′ (SEQ ID NO: 6); (4) Bcl-xL forward, 5′-CCATACTGAGGGACCAACTG-3′ (SEQ ID NO: 7); reverse, 5′-GGCTGCTCTTGTAGGAAGTG-3′ (SEQ ID NO: 8); (5) p21 forward, 5′-TGTCTTTCCTGGCACTAACG-3′ (SEQ ID NO: 9); reverse, 5′-AAACAGTCCAGGCCAGTATG-3′ (SEQ ID NO: 10); (6) IL-8 forward, 5′-GTCTGCTAGCCAGGATCCAC-3′ (SEQ ID NO: 11); reverse, 5′-GCTTCCACATGTCCTCACAA-3′ (SEQ ID NO: 12); (7) MMP-3, forward, 5′-TCTGAGGGGAGAAATCCTGA-3′ (SEQ ID NO: 13); reverse, 5′-GGAAGAGATGGCCAAAATGA-3′ (SEQ ID NO: 14); (8) Cyclin A2, forward, 5′-ATGGACCTTCACCAGACCTA-3′ (SEQ ID NO: 15); reverse, 5′-TGGGTTGAGGAGAGAAACAC-3′ (SEQ ID NO: 16); (9) CDK1 forward, 5′-AGCCGGGATCTACCATAC-3′ (SEQ ID NO: 17); reverse, 5′-TCATGGCTACCACTTGAC-3′ (SEQ ID NO: 18); (10) IL-10 forward, 5′-GCTGCTCTGGGATTCTCTTC-3′ (SEQ ID NO: 19); reverse, 5′-TGGCGAGCTCAGGTACTTC-3′ (SEQ ID NO: 20).

Viability Assay:

Growing and senescent cells were plated in 12-well plates at 7.5×10⁴ cells per well. The following day, cells were treated with DMSO control, ABT-737 (Selleckchem, USA) or ABT-199 (ChemieTek, USA) and cell viability was analyzed 24 hours thereafter. 300 μl of PrestoBlue Reagent (Invitrogen, USA) were added to each well, and plates were incubated for 20 minutes at 37 C. 100 μl samples were taken in duplicates to a 96 well plate and read at an OD of 540 nm using a Tecan plate reader (Infinite® M200). A three-hour pre incubation with 100 μM z-VAD-fmk (Santa Cruz, USA) was performed prior to the addition of ABT-737 where indicated.

siRNA:

ON-TARGETplus SMARTpool small-interfering RNAs targeting p21, Bcl-2, Bcl-w, Bcl-xL and the nontargeting pool siRNAs (control) were transfected into cells with the Dharmafect 1 reagent (all from Dharmacon, Lafayette, Colo., USA). siRNAs were washed away 24 hours post transfection and viability was analyzed four days thereafter as described above.

### Example 1

### Expression of Bcl-w and Bcl-xL Proteins is Elevated in Senescent Cells

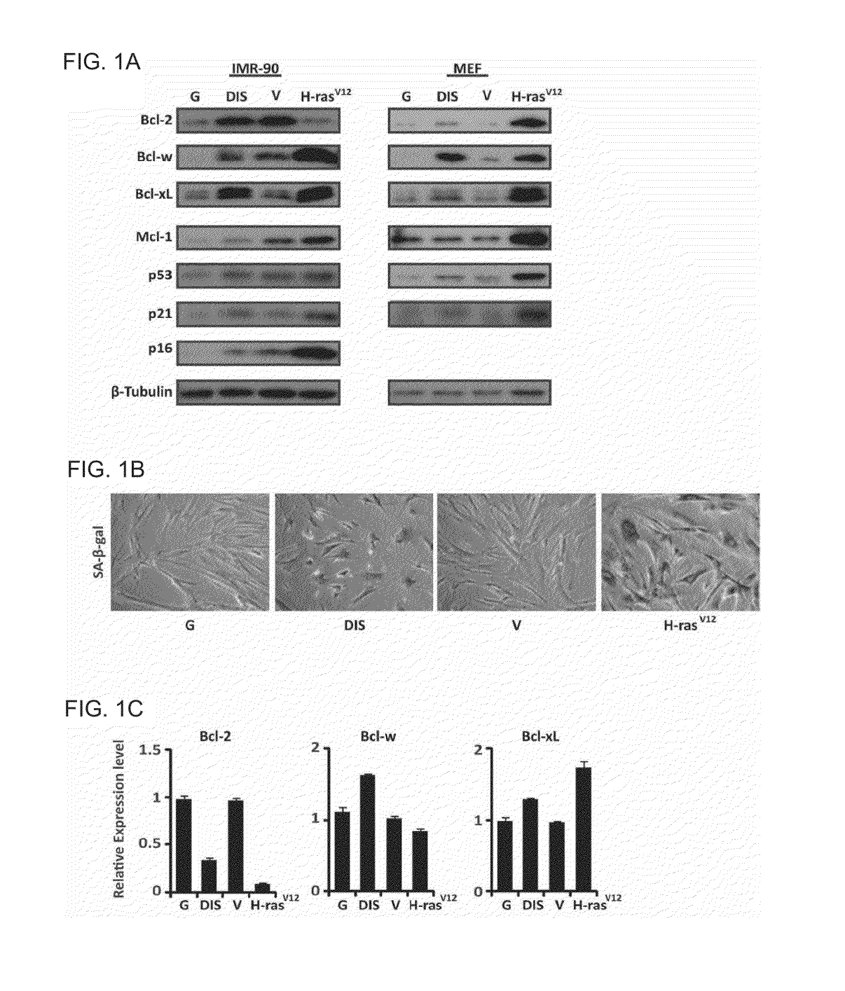

Protein levels of Bcl-2 family members in growing and senescent normal human (IMR-90) and mouse (MEF) diploid fibroblast cells were analyzed. Senescence was induced in these cells either by expression of oncogenic H-ras^{v12} or by treatment with the DNA damaging agent Etoposide. Bcl-w and Bcl-xL levels were elevated in senescent cells of both human and mouse origin. This effect was unrelated to the stimulus that was used to induce senescence (FIG. 1A). In contrast, the changes in Mcl-1 and Bcl-2 levels were either less pronounced or dependent on cell origin and the stress stimuli used to induce senescence. The levels of classical markers of senescence p16, p21 or p53, serve us as positive controls for senescent phenotype of the cells, together with positive SA-β-gal staining (FIG. 1A-B). Of note, the levels of mRNA of these genes were not considerably altered between growing and senescent cells, indicating that the increase in the protein levels might be regulated at the post-transcription level (FIG. 1C).

### Example 2

### Combined Knockdown of Bcl-w and Bcl-xL Induces Death of Senescent Cells

In order to distinguish which of the three proteins, Bcl-2, Bcl-w and Bcl-xL provide the apoptotic resistance of Etoposide treated senescent IMR-90 cells, the present inventors attempted to specifically inhibit the function of each of these proteins individually. siRNA was used to knock down Bcl-w and Bcl-xL, and a specific inhibitor, ABT-199, to block Bcl-2 as siRNA was ineffective in knocking down this gene on the protein level (FIG. 2B). Knocking down Bcl-w and Bcl-xL separately in senescent cells led to minor reduction in their viability (FIG. 3A). Interestingly, a combined knock-down had a synergistic effect, bringing about the demise of 50% of the cells. To assess the additive contribution of Bcl-2 inhibition on top of Bcl-w and Bcl-xL knockdown, the Bcl-2 inhibitor, ABT-199 was used (FIG. 3B). Inhibition of Bcl-2 had a statistically significant, but minor additive effect to that of Bcl-w and Bcl-xL on senescent cell viability. In addition, inhibition of Bcl-2 alone with ABT-199 had almost no effect the viability of senescent cells besides a minor decrease in cell viability for oncogenic H-ras^{v12} induced senescent IMR-90 cells (FIG. 3C-D).

### Example 3

### The BH3 Mimetic ABT-737 Induces Cell Death of Senescent Cells

To further test the hypothesis that an increase in the levels of anti-apoptotic proteins Bcl-w and Bcl-xL could account for the apoptotic resistance of senescent cells by an independent approach, cells were treated with the pharmacological inhibitor of the Bcl-2 family of proteins, ABT-737 (Chauhan et al., 2007). Normal human (IMR-90) and mouse (MEF) fibroblasts were induced to undergo senescence by the induction of DNA damage or by transduction with oncogenic H-Ras^{V12}. After the senescence phenotype had been established, cells were treated with ABT-737 for 24 hours. Growing, vehicle treated, or vector transduced cells served as control for DNA damage or oncogenic H-Ras^{v12} respectively. This treatment reduced the viability of senescent cells by 50%, while only having a minor effect on control cells (FIGS. 4A-B). Therefore, it has been shown that pharmacological inhibition of Bcl-2 family proteins leads to specific elimination of senescent cells.

### Example 4

### ABT-737 Kills Senescent Cells Via Caspase-Dependent Apoptosis

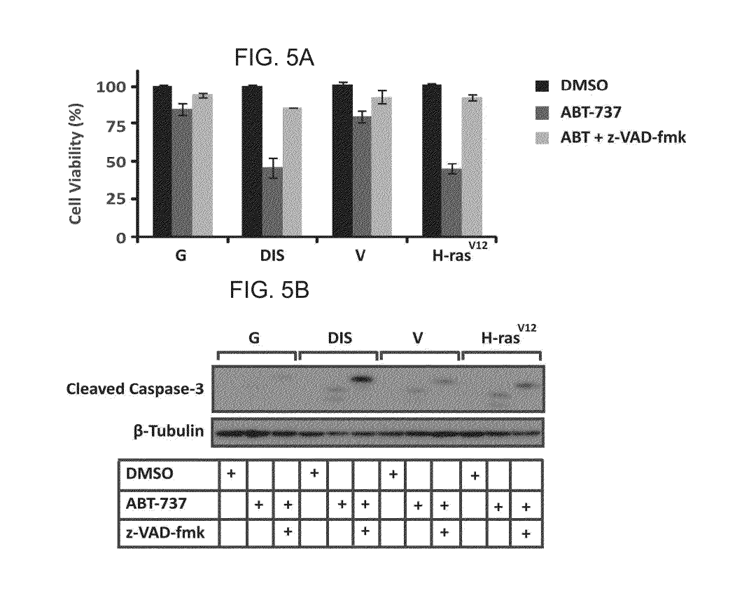

Members of the Bcl-2 family negatively regulate apoptotic pathway (Azmi et al., 2011; Cory et al., 2003; Reed, 2008). To determine whether ABT-737 kills senescent cells via the apoptotic pathway, DNA damage induced senescent IMR-90 cells, oncogenic H-Ras^{v12} induced senescent cells and control growing cells were treated with ABT-737 alone or in combination with pan-caspase inhibitor z-VAD-fmk. As expected, ABT-737 induced death in senescent cells while z-VAD-fmk prevented death of the cells following ABT-737 treatment (FIG. 5A). Since caspase-3 is cleaved by the apoptotic machinery, the present inventors examined the presence of its activated cleaved form following treatment with ABT-737 alone or in combination with pan-caspase inhibitor z-VAD-fmk. Only senescent cells treated with ABT-737 show caspase-3 cleavage (FIG. 5B). The addition of z-VAD-fmk abolished this cleavage. It may be concluded that ABT-737 induces apoptosis in senescent cells.

### Example 5

### p21 (CDKN1A) Maintains the Viability of Senescent Cells

As an inhibitor of CDK4 and CDK2, p21 is a main regulator of cellular senescence (Campisi and d′Adda di Fagagna, 2007). It was also suggested to inhibit apoptosis in some circumstances (Abbas and Dutta, 2009). To explore the contribution of p21 to the viability of senescent cells it was knocked down using siRNA in growing and senescent cells normal human (IMR-90, BJ) and mouse (MEF) fibroblasts as well as in lung cancer cells (H1299). Whereas p21 knockdown in growing cells had no deleterious effect on cell viability, its knockdown in senescent cells led to a 30%, 50%, 75% and 30% reduction in cell viability for IMR-90, BJ, H1299 and MEF cells respectively (FIGS. 6A-D). Interestingly, continuous reduction in the viability of senescent BJ cells transfected with siRNA for p21 was observed over time, indicative of a cumulative effect for p21 knockdown (FIG. 6E). Therefore, p21 is necessary to maintain the viability of senescent cells.

### Example 6

### The Death of Senescent Cells is p53- and pRB-Independent and Involves Caspase-3 Activation

Stimuli that generate a DNA damage response (for example, ionizing radiation and telomere dysfunction) induce senescence primarily through the p53 pathway (Campisi and d′Adda di Fagagna, 2007). Active p53 establishes the senescence growth arrest in part by inducing the expression of p21, a cyclin-dependent kinase (CDK) inhibitor that, among other activities, suppresses the phosphorylation and, hence, the inactivation of pRB. pRB halts cell proliferation by suppressing the activity of E2F, a transcription factor that stimulates the expression of genes that are required for cell-cycle progression.

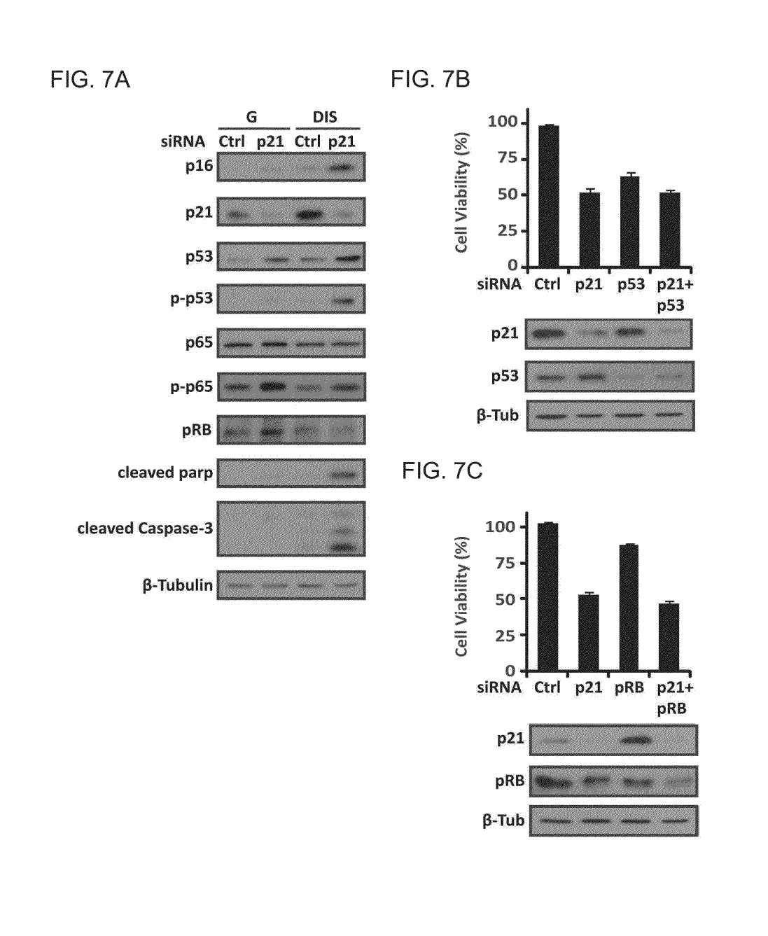

Knocking down p21 in senescent but not in growing BJ cells led to activation of the apoptosis machinery indicative from cleavage of apoptosis effectors caspase-3 and PARP. This was accompanied by a pronounced increase in p53 level and activity (p-p53) and a reduction in pRB level (FIG. 7A). As p53 and E2F are known to induce apoptosis in response to DNA damage, we checked whether p21 knockdown affects the viability of senescent cells in a p53 or pRB-dependent manner.

p21, p53 or pRB were knocked-down individually or in combination with p21, in Etoposide treated BJ cells. Surprisingly, p53 knockdown reduced the viability of senescent cells, but to a lesser extend compare to p21 alone (FIG. 7B).When both genes were knocked-down simultaneously, no additive effect for p53 was detected, comparing to p21 knockdown alone. As seen from the western blot, p53 knockdown lead to a decrease in p21 level. Thus it may be concluded that p21 level is responsible for maintaining the viability of senescent cells downstream of p53, rather than p53 itself.

The knockdown of pRB together with p21 had no additive effect on the viability of senescent cells comparing to the knockdown of p21 alone (FIG. 7C). Surprisingly, when pRB was knocked-down alone it had no effect on the viability of the cells, but rather caused an increase in p21 level. Therefore, p21 maintains the viability of senescent cells in a p53 and pRB independent manner.

### Example 7

### Death of Senescent Cells Following p21 Knockdown is Only Partially Caspase-Dependent

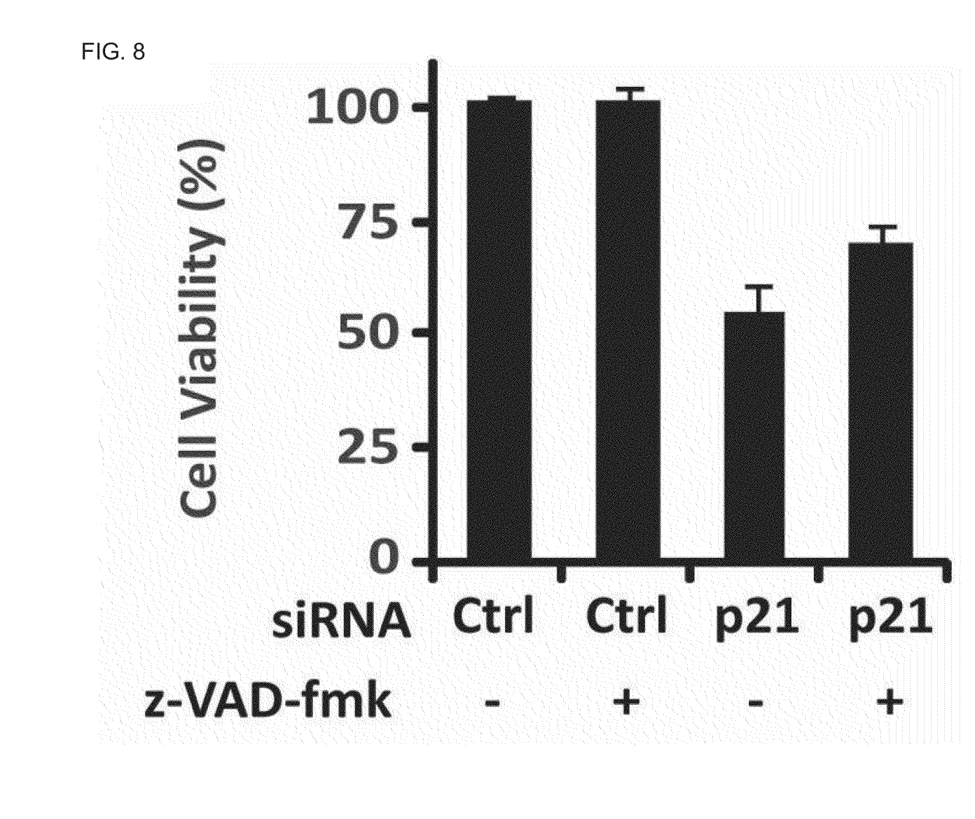

To elucidate the type of cell death caused by p21 knockdown, caspase mediated cell death was evaluated by addition of the pan-Caspase inhibitor, z-VAD-fmk. z-VAD-fmk was able to rescue cell death by only 20% (FIG. 8). Caspase-3 was activated in Etoposide treated cells with p21 knockdown, as cleaved caspase-3 and cleaved PARP bands are evident (FIG. 8). Given that z-VAD-fmk was not able to completely rescue the reduction in cell viability mediated by p21 knockdown, it may be reasoned that caspase-dependent apoptosis is only partially responsible for the cell death observed. Therefore, it may be hypothesized that other cell death mechanisms, such as Necroptosis, might be induced by the knockdown of p21 in senescent cells.

### Example 8

### E2F Targets and Inflammation Genes are Upregulated as a Response to p21 Knockdown

p21 can inhibit the transcriptional activity of the transcription factors such as E2F1, STAT3 and MYC through direct binding and inhibition of their trans activation activity (Abbas and Dutta, 2009). Therefore changes in mRNA levels of E2F targets as well as SASP components were measured in response to p21 knockdown. Significant increase in mRNA levels of the E2F targets Cyclin-A2 and CDK-1 were detected following p21 knockdown (FIGS. 9A-E). In addition, p21 knockdown led to increase in IL-8 and IL-1β mRNA levels which might point towards an inflammatory response linked to senescent cell death. Therefore, p21 knockdown induces pro-inflammatory response and cell death in senescent cells. This approach might lead to increase in the therapeutic potential of this approach because the inflammatory cytokines will recruit the immune system to kill the cells that were not eliminated by the knockdown itself.

### Example 9

### p21 Knockdown Reduces Liver Fibrosis

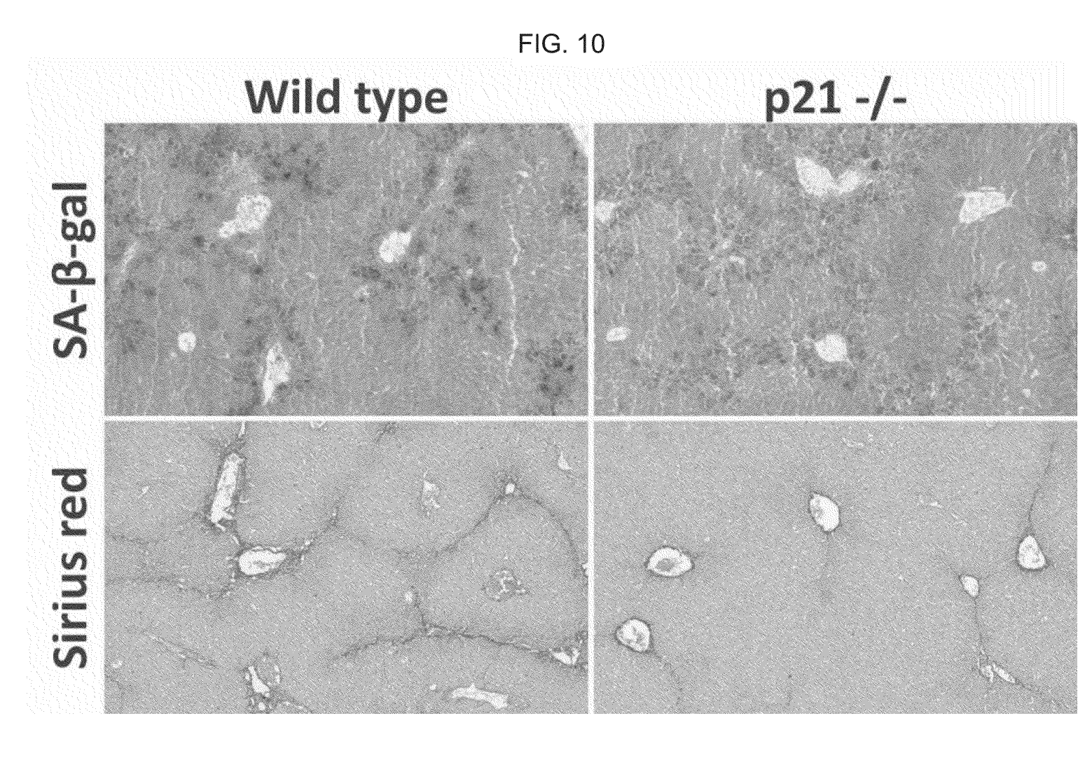

In the fibrotic liver, senescent cells are derived primarily from activated hepatic stellate cells, which initially proliferate in response to liver damage and produce the extracellular matrix deposited in the fibrotic scar (Krizhanovsky et al, Cell, 2008). To evaluate the effect of elimination of senescent cells by means of p21 knockdown on liver fibrosis, the present inventors induced fibrosis in wild type and p21−/− mice. The mice were subjected to 6 week treatment with CCl₄, to induce liver fibrosis as described previously (Krizhanovsky et al, Cell 2008). Following the treatment, livers from mice of both genotypes were tested for the presence of senescent cells by SA-β-gal staining and for the degree of fibrosis by Sirius red staining. In concordance with the tissue culture experiments, livers derived from p21 knockout mice contained significantly less senescent cells relative to wild type (FIG. 10). Importantly, this reduction was accompanied by a significant reduction in the amount of the fibrotic scar (FIG. 10).

These observations suggest that in vivo, in the absence of p21 the frequency of senescent cells declines and leads to decrease in fibrosis. Therefore, the present inventors propose that elimination of senescent cells by means of p21 inhibition might have a therapeutic effect on fibrosis.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### BIBLIOGRAPHY

Abbas, T., and Dutta, A. (2009). p21 in cancer: intricate networks and multiple activities. Nat Rev Cancer 9, 400-414.
Acosta, J. C., and Gil, J. (2012). Senescence: a new weapon for cancer therapy. Trends Cell Biol 22, 211-219.
Adams, P. D. (2009). Healing and hurting: molecular mechanisms, functions, and pathologies of cellular senescence. Mol Cell 36, 2-14.
Azmi, A. S., Wang, Z., Philip, P. A., Mohammad, R. M., and Sarkar, F. H. (2011). Emerging Bcl-2 inhibitors for the treatment of cancer. Expert opinion on emerging drugs 16, 59-70.
Baker, D. J., Wijshake, T., Tchkonia, T., LeBrasseur, N. K., Childs, B. G., van de Sluis, B., Kirkland, J. L., and van Deursen, J. M. (2011). Clearance of p16Ink4a-positive senescent cells delays ageing-associated disorders. Nature 479, 232-236.
Braig, M., Lee, S., Loddenkemper, C., Rudolph, C., Peters, A. H., Schlegelberger, B., Stein, H., Dorken, B., Jenuwein, T., and Schmitt, C. A. (2005). Oncogene-induced senescence as an initial barrier in lymphoma development. Nature 436, 660-665.
Campisi, J. (2011). Cellular senescence: putting the paradoxes in perspective. Curr Opin Genet Dev 21, 107-112.
Campisi, J., and d′Adda di Fagagna, F. (2007). Cellular senescence: when bad things happen to good cells. Nat Rev Mol Cell Biol 8, 729-740.
Chan, H. M., Narita, M., Lowe, S. W., and Livingston, D. M. (2005). The p400 E1A-associated protein is a novel component of the p53->p21 senescence pathway. Genes Dev 19, 196-201.
Chauhan, D., Velankar, M., Brahmandam, M., Hideshima, T., Podar, K., Richardson, P., Schlossman, R., Ghobrial, I., Raje, N., Munshi, N., et al. (2007). A novel Bcl-2/Bcl-X(L)/Bcl-w inhibitor ABT-737 as therapy in multiple myeloma. Oncogene 26, 2374-2380.
Collado, M., Blasco, M. A., and Serrano, M. (2007). Cellular senescence in cancer and aging. Cell 130, 223-233.
Collado, M., and Serrano, M. (2010). Senescence in tumours: evidence from mice and humans. Nat Rev Cancer 10, 51-57.
Cory, S., Huang, D. C., and Adams, J. M. (2003). The Bcl-2 family: roles in cell survival and oncogenesis. Oncogene 22, 8590-8607.
Kortlever, R. M., and Bernards, R. (2006). Senescence, wound healing and cancer: the PAI-1 connection. Cell Cycle 5, 2697-2703.
Krizhanovsky, V., Xue, W., Zender, L., Yon, M., Hernando, E., and Lowe, S. W. (2008a). Implications of cellular senescence in tissue damage response, tumor suppression, and stem cell biology. Cold Spring Harb Symp Quant Biol 73, 513-522.
Krizhanovsky, V., Yon, M., Dickins, R. A., Hearn, S., Simon, J., Miething, C., Yee, H., Zender, L., and Lowe, S. W. (2008b). Senescence of activated stellate cells limits liver fibrosis. Cell 134, 657-667.
Krtolica, A., Parrinello, S., Lockett, S., Desprez, P. Y., and Campisi, J. (2001). Senescent fibroblasts promote epithelial cell growth and tumorigenesis: a link between cancer and aging. Proc Natl Acad Sci USA 98, 12072-12077.
Kuilman, T., Michaloglou, C., Mooi, W. J., and Peeper, D. S. (2010). The essence of senescence. Genes Dev 24, 2463-2479.
Marcotte, R., Lacelle, C., and Wang, E. (2004). Senescent fibroblasts resist apoptosis by downregulating caspase-3. Mech Ageing Dev 125, 777-783.
Michaloglou, C., Vredeveld, L. C., Soengas, M. S., Denoyelle, C., Kuilman, T., van der Horst, C. M., Majoor, D. M., Shay, J. W., Mooi, W. J., and Peeper, D. S. (2005). BRAFE600-associated senescence-like cell cycle arrest of human naevi. Nature 436, 720-724.
Murata, Y., Wakoh, T., Uekawa, N., Sugimoto, M., Asai, A., Miyazaki, T., and Maruyama, M. (2006). Death-associated protein 3 regulates cellular senescence through oxidative stress response. FEBS Lett 580, 6093-6099.
Narita, M., Krizhanovsky, V., Nunez, S., Chicas, A., Hearn, S. A., Myers, M. P., and Lowe, S. W. (2006). A novel role for high-mobility group a proteins in cellular senescence and heterochromatin formation. Cell 126, 503-514.
Narita, M., Nunez, S., Heard, E., Lin, A. W., Hearn, S. A., Spector, D. L., Hannon, G. J., and Lowe, S. W. (2003). Rb-mediated heterochromatin formation and silencing of E2F target genes during cellular senescence. Cell 113, 703-716.
Naylor, R. M., Baker, D. J., and van Deursen, J. M. (2013). Senescent cells: a novel therapeutic target for aging and age-related diseases. Clinical pharmacology and therapeutics 93, 105-116.
Reed, J. C. (2008). Bcl-2-family proteins and hematologic malignancies: history and future prospects. Blood 111, 3322-3330.
Sagiv, A., Biran, A., Yon, M., Simon, J., Lowe, S. W., and Krizhanovsky, V. (2012). Granule exocytosis mediates immune surveillance of senescent cells. Oncogene DOI:10.1038/onc.2012.206.
Schmitt, C. A., Fridman, J. S., Yang, M., Lee, S., Baranov, E., Hoffman, R. M., and Lowe, S. W. (2002). A senescence program controlled by p53 and p16INK4a contributes to the outcome of cancer therapy. Cell 109, 335-346.
Uraoka, M., Ikeda, K., Kurimoto-Nakano, R., Nakagawa, Y., Koide, M., Akakabe, Y., Kitamura, Y., Ueyama, T., Matoba, S., Yamada, H., et al. (2011). Loss of bcl-2 during the senescence exacerbates the impaired angiogenic functions in endothelial cells by deteriorating the mitochondrial redox state. Hypertension 58, 254-263.
Wang, E. (1995). Senescent human fibroblasts resist programmed cell death, and failure to suppress bcl2 is involved. Cancer Res 55, 2284-2292.
Xue, W., Zender, L., Miething, C., Dickins, R. A., Hernando, E., Krizhanovsky, V., Cordon-Cardo, C., and Lowe, S. W. (2007). Senescence and tumour clearance is triggered by p53 restoration in murine liver carcinomas. Nature 445, 656-660.
Zeitlin, B. D., Zeitlin, I. J., and Nor, J. E. (2008). Expanding circle of inhibition: small-molecule inhibitors of Bcl-2 as anticancer cell and antiangiogenic agents. J Clin Oncol 26, 4180-4188.

1. A method of treating an inflammatory or fibrotic disease in a subject in need thereof comprising administering to the subject a therapeutically effective amount of an agent which down-regulates an activity and/or an amount of (Bcl-xL) and/or Bcl-w, thereby treating the inflammatory or fibrotic disease, with the proviso that the inflammatory disease is not cancer.

2. The method of claim 1, wherein the agent is a chemical agent.

3. The method of claim 1, wherein the agent is a polynucleotide agent targeted against the Bcl-xL and/or Bcl-w.

4. The method of claim 1, wherein said disease is associated with cartilage degeneration.

5. The method of claim 1,
wherein said disease is selected from the group consisting of liver fibrosis, wound healing, skin fibrosis, pulmonary disease, kidney fibrosis, prostatitis, atherosclerosis, arthritis and pancreatitis.

6. An article of manufacture comprising:
(i) an agent which down-regulates an activity and/or an amount of Bcl-xL and/or Bcl-w; and
(ii) an agent which down-regulates an activity and/or an amount of p21.

7-8. (canceled)

9. The article of manufacture of claim 6 further comprising at least one agent selected from the group consisting of a sebum-regulating agent, an antibacterial and/or antifungal agent, a keratolytic agent and/or keratoregulating agent, an astringent, an anti-inflammatory and/or anti-irritant, an antioxidant and/or free-radical scavenger, a cicatrizing agent, an anti-aging agent and a moisturizing agent.

10. (canceled)

11. A pharmaceutical composition comprising a pharmaceutically acceptable carrier and as an active agent:
(i) an agent which down-regulates an activity and/or an amount of Bcl-xL and/or Bcl-w; and
(ii) an agent which down-regulates an activity and/or an amount of p21.

12. The pharmaceutical composition of claim 11, formulated for topical delivery.

13-15. (canceled)

16. A composition comprising a carrier and at least one active agent which down-regulates an activity and/or an amount of p21 and at least one active agent which down-regulates an activity and/or an amount of Bcl-xL and/or Bcl-w, wherein the composition is formulated for topical administration.

17-19. (canceled)

20. A method of treating an inflammatory or fibrotic disease in a subject in need thereof comprising administering to the subject a therapeutically effective amount of an agent which down-regulates an activity and/or an amount of cyclin-dependent kinase inhibitor 1 (p21), thereby treating the inflammatory or fibrotic disease, with the proviso that the disease is not cancer.

21. The method of claim 20, wherein said agent is a polynucleotide directed to an endogenous nucleic acid sequence expressing said p21.

22. (canceled)

23. The method of claim 20, further comprising administering to the subject at least one agent which down-regulates an activity and/or an amount of Bcl-xL and/or Bcl-w.

24-27. (canceled)

28. The method of claim 20, wherein said disease is associated with cartilage degeneration.

29. The method of claim 20, wherein said disease is selected from the group consisting of liver fibrosis, wound healing, skin fibrosis, pulmonary disease, osteoporosis, kidney fibrosis, prostatitis, atherosclerosis, arthritis and pancreatitis.

30. The method of claim 29, wherein said pulmonary disease comprises chronic obstructive pulmonary disease (COPD).

31-37. (canceled)

38. A method of treating a pre-malignant lesion in a subject in need thereof comprising administering to the subject a therapeutically effective amount of an agent which down-regulates an activity and/or an amount of Bcl-xL and/or Bcl-w and/or cyclin-dependent kinase inhibitor 1 (p21), thereby treating the pre-malignant lesion.

39-40. (canceled)


SEQUENCE LISTING (37 sequences)

SEQ ID NO: 1 - DNA, 19 bp, Artificial sequence
  Single strand DNA oligonucleotide
gacagtcagccgcatcttc

SEQ ID NO: 2 - DNA, 19 bp, Artificial sequence
  Single strand DNA oligonucleotide
cgttgactccgaccttcac

SEQ ID NO: 3 - DNA, 24 bp, Artificial sequence
  Single strand DNA oligonucleotide
actggagagtgctgaagattgatg

SEQ ID NO: 4 - DNA, 31 bp, Artificial sequence
  Single strand DNA oligonucleotide
ctacttcctctgtgatgttgtattttttaag

SEQ ID NO: 5 - DNA, 20 bp, Artificial sequence
  Single strand DNA oligonucleotide
tgacacctgggtggaaagag

SEQ ID NO: 6 - DNA, 20 bp, Artificial sequence
  Single strand DNA oligonucleotide
ccactgtggtcccatctaag

SEQ ID NO: 7 - DNA, 20 bp, Artificial sequence
  Single strand DNA oligonucleotide
ccatactgagggaccaactg

SEQ ID NO: 8 - DNA, 20 bp, Artificial sequence
  Single strand DNA oligonucleotide
ggctgctcttgtaggaagtg

SEQ ID NO: 9 - DNA, 20 bp, Artificial sequence
  Single strand DNA oligonucleotide
tgtctttcctggcactaacg

SEQ ID NO: 10 - DNA, 20 bp, Artificial sequence
  Single strand DNA oligonucleotide
aaacagtccaggccagtatg

SEQ ID NO: 11 - DNA, 20 bp, Artificial sequence
  Single strand DNA oligonucleotide
gtctgctagccaggatccac

SEQ ID NO: 12 - DNA, 20 bp, Artificial sequence
  Single strand DNA oligonucleotide
gcttccacatgtcctcacaa

SEQ ID NO: 13 - DNA, 20 bp, Artificial sequence
  Single strand DNA oligonucleotide
tctgaggggagaaatcctga

SEQ ID NO: 14 - DNA, 20 bp, Artificial sequence
  Single strand DNA oligonucleotide
ggaagagatggccaaaatga

SEQ ID NO: 15 - DNA, 20 bp, Artificial sequence
  Single strand DNA oligonucleotide
atggaccttcaccagaccta

SEQ ID NO: 16 - DNA, 20 bp, Artificial sequence
  Single strand DNA oligonucleotide
tgggttgaggagagaaacac

SEQ ID NO: 17 - DNA, 18 bp, Artificial sequence
  Single strand DNA oligonucleotide
agccgggatctaccatac

SEQ ID NO: 18 - DNA, 18 bp, Artificial sequence
  Single strand DNA oligonucleotide
tcatggctaccacttgac

SEQ ID NO: 19 - DNA, 20 bp, Artificial sequence
  Single strand DNA oligonucleotide
gctgctctgggattctcttc

SEQ ID NO: 20 - DNA, 19 bp, Artificial sequence
  Single strand DNA oligonucleotide
tggcgagctcaggtacttc

SEQ ID NO: 21 - protein, 233 aa, Homo sapiens
MSQSNRELVVDFLSYKLSQKGYSWSQFSDVEENRTEAPEGTESEMETPSAINGNPSWHLADSPAVNGATGHSSSLDAREVIPMAAVKQALREAGDEFELRYRRAFSDLTSQLHITPGTAYQSFEQVVNELFRDGVNWGRIVAFFSFGGALCVESVDKEMQVLVSRIAAWMATYLNDHLEPWIQENGGWDTFVELYGNNAAAESRKGQERFNRWFLTGMTVAGVVLLGSLFSRK

SEQ ID NO: 22 - DNA, 2575 bp, Homo sapiens
ggaggaggaagcaagcgagggggctggttcctgagcttcgcaattcctgtgtcgccttctgggctcccagcctgccgggtcgcatgatccctccggccggagctggtttttttgccagccaccgcgaggccggctgagttaccggcatccccgcagccacctcctctcccgacctgtgatacaaaagatcttccgggggctgcacctgcctgcctttgcctaaggcggatttgaatctctttctctcccttcagaatcttatcttggctttggatcttagaagagaatcactaaccagagacgagactcagtgagtgagcaggtgttttggacaatggactggttgagcccatccctattataaaaatgtctcagagcaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactggccacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtaaactggggtcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatgcagcagccgagagccgaaagggccaggaacgcttcaaccgctggttcctgacgggcatgactgtggccggcgtggttctgctgggctcactcttcagtcggaaatgaccagacactgaccatccactctaccctcccacccccttctctgctccaccacatcctccgtccagccgccattgccaccaggagaaccactacatgcagcccatgcccacctgcccatcacagggttgggcccagatctggtcccttgcagctagttttctagaatttatcacacttctgtgagacccccacacctcagttcccttggcctcagaattcacaaaatttccacaaaatctgtccaaaggaggctggcaggtatggaagggtttgtggctgggggcaggagggccctacctgattggtgcaacccttaccccttagcctccctgaaaatgtttttctgccagggagcttgaaagttttcagaacctcttccccagaaaggagactagattgcctttgttttgatgtttgtggcctcagaattgatcattttccccccactctccccacactaacctgggttccctttccttccatccctaccccctaagagccatttaggggccacttttgactagggattcaggctgcttgggataaagatgcaaggaccaggactccctcctcacctctggactggctagagtcctcactcccagtccaaatgtcctccagaagcctctggctagaggccagccccacccaggagggagggggctatagctacaggaagcaccccatgccaaagctagggtggcccttgcagttcagcaccaccctagtcccttcccctccctggctcccatgaccatactgagggaccaactgggcccaagacagatgccccagagctgtttatggcctcagctgcctcacttcctacaagagcagcctgtggcatctttgccttgggctgctcctcatggtgggttcaggggactcagccctgaggtgaaagggagctatcaggaacagctatgggagccccagggtcttccctacctcaggcaggaagggcaggaaggagagcctgctgcatggggtggggtagggctgactagaagggccagtcctgcctggccaggcagatctgtgccccatgcctgtccagcctgggcagccaggctgccaaggccagagtggcctggccaggagctcttcaggcctccctctctcttctgctccacccttggcctgtctcatccccaggggtcccagccaccccgggctctctgctgtacatatttgagactagtttttattccttgtgaagatgatatactatttttgttaagcgtgtctgtatttatgtgtgaggagctgctggcttgcagtgcgcgtgcacgtggagagctggtgcccggagattggacggcctgatgctccctcccctgccctggtccagggaagctggccgagggtcctggctcctgaggggcatctgcccctcccccaacccccaccccacacttgttccagctctttgaaatagtctgtgtgaaggtgaaagtgcagttcagtaataaactgtgtttactcagtgaaaaaaaaaaaaaaaaaa

SEQ ID NO: 23 - protein, 193 aa, Homo sapiens
MATPASAPDTRALVADFVGYKLRQKGYVCGAGPGEGPAADPLHQAMRAAGDEFETRFRRTFSDLAAQLHVTPGSAQQRFTQVSDELFQGGPNWGRLVAFFVFGAALCAESVNKEMEPLVGQVQEWMVAYLETRLADWIHSSGGWAEFTALYGDGALEEARRLREGNWASVRTVLTGAVALGALVTVGAFFASK

SEQ ID NO: 24 - DNA, 3621 bp, Homo sapiens
atcacccggcgccgggccctccctctgcccccccctttctcctccctccttccttccctcccttcctccctctctccctccctcccagctcctgcaccaggaaacggcccggatcccggcagcggcctgacccggctccacgctggccaggaggatgaaaggccccagctgggggctccttgccaccagtgctgtgtcttaagagctgccatcccggctggccgcccggatggcgaccccagcctcggccccagacacacgggctctggtggcagactttgtaggttataagctgaggcagaagggttatgtctgtggagctggccccggggagggcccagcagctgacccgctgcaccaagccatgcgggcagctggagatgagttcgagacccgcttccggcgcaccttctctgatctggcggctcagctgcatgtgaccccaggctcagcccaacaacgcttcacccaggtctccgatgaactttttcaagggggccccaactggggccgccttgtagccttctttgtctttggggctgcactgtgtgctgagagtgtcaacaaggagatggaaccactggtgggacaagtgcaggagtggatggtggcctacctggagacgcggctggctgactggatccacagcagtgggggctgggcggagttcacagctctatacggggacggggccctggaggaggcgcggcgtctgcgggaggggaactgggcatcagtgaggacagtgctgacgggggccgtggcactgggggccctggtaactgtaggggccttttttgctagcaagtgaaagtccagggccaggtggggctaggtgtggctgggggccaggagagcaggaacagaacagagaaatgcccttggaagaagtggagttggtggatgggtgggcatggaacaggatgggcagagaaagggtagtgtgtgagggagctgagtaggccaggtaggcgattggaagagtgagcaggacacagaggggaggggaatgttttggcaagtttaggggcacaggagatgtagtcgttccagggctgggggaggtgggagggatcacgcctataggtgtgggcacatgaaacgacctggaacttgcttcacagccctgaggaaggtggacttacataagcagctgtattccattagatgagtgggatttagggaacgcagaaggcacatccctttggaatggaagcttaggggttctcaggtgatagggagaggtggctgttaacagtgggctgcttggacacgcgtgtgcatgtgcacgcatgctggtgtgcatgctgggctgcctggcaaatctggtggtgatgggattcctcaaggagaaaacattccctcttgcaatggcaagaactaggggcagttctctgtccctcctcccaacccctcctttcccctgcccttgtcctgatgcctcaaggcttagagagaaacattgtatccagaccgagggctctgctgcttctttccagaaagtgattggcaaggctttggagagaagagcagttctgcagctggccttgttccttcatcatcccccttccttgtgcattatgcacttgctgctgcctcctgggctctgatagaagggcagggctgttgagcctggatgggtggaggcttaggtagccggacctgcctgccaccctcctctcccactcaggcacaatggtgcctaaagtgtttccaatctctgggacctctgtacccaaactgaaactctaaattggggccctaactaattttccttttgaggttgtgggcataagtgctgatctagaatacagtctgggtcccacactgtgtctcagtgagactgttgatgccttgagatgaccatttcagatctgaatcccatgggtgtgagggtgatgggtactccaggactggcctatgctgtgttgtgggctttggttcggctttatcaggggccaggcatatgggttctagagtacctaccatgacctagaagcatttatgatttatttgaagccacactgtttgcatgggtgttacttgtctgtacctcagagtctgaggatgttaactttggaactcgcagtcctctagaacagcttcagattatggctttttcttttgaggaagaaattattcactccagatgcatgccctgagccagacctcactgctgcactttccaaggtgctaagattgctgctctccaatgctaactttctgacacagtgctctagaaccctgcctgtggtcctgagcactgatcaccttagctagaccatggttgactcttcttggagattttcacttggtcctagaatgtggcaacgtagttgtgctcgccagaacgtgggaccaaattggcctcaggtgttgagtccagacttctgcttttgagagagggctgcactttttcatggtatttctaggggaggtggtaggctgcatgtgccacttggtcttgttgtgagtatgctgacaccagaaactcagagccagcttgtggcaagcagttggggtggggggtctctgacttgctcaggacaaactaggccagtggttttcaaactgcttggcagagccctgaagtttcctaggggttgcctcaggagtccttggggagatgaagggggtggggagctgagcaggctgggcaatttgccctcaaacagaacagctccccttgtagctgtcttacatattggggttcagggtaagattttatttgcattaaggggtttgctgctgaaaaaaagttggaaaaccactgactagaccatcggctccaaattggagtctgtgcttccttccccaggtatggagcacactcttcaccctaccctctaccacaggacacatatccctgttagcattccccgggacctttagccaagaggagctgcagggaccatggccaggttaccaaaatgccctgctctgaagccttgacacctgggtggaaagagaggctgttttctgaaagggtaaagggcttggtctggattcccagaagcatagcttagatgggaccacagtgggcaattttgacctgtcctgcccttcttagcttgaagggaaaccccagagactcttctgtcagggaaaactagggactctcttctagagccatatagttccttgggattagctcttggccaagaaggctgagtatggttcccaatttttaaatccatttcattttttaaaaaataagggaaataaatgtaattgccatttttcaaagattaagtaggaggagaggggtttcttgctctccagagcccaaagggacaaatagggactttgtttaggccaaggaaggagcggaagtagggcaactcggtcctgcgattattaatcccactccccacttattctagggcacacaaacactattttacttttttaaaatcataaaacggcagaacagatttggttagtttagaagaaaagaaagctctataaatataaatctatattcctgtatttttatttaataatttataaataccaagttcatttgacttttatttttgtgtaatatgtaatgatcgtattaaaaacaataaataaagcccagaagtttaatgagaaggactgaaaaaaaaaaaaaaaaaa

SEQ ID NO: 25 - protein, 164 aa, Homo sapiens
MSEPAGDVRQNPCGSKACRRLFGPVDSEQLSRDCDALMAGCIQEARERWNFDFVTETPLEGDFAWERVRGLGLPKLYLPTGPRRGRDELGGGRRPGTSPALLQGTAEEDHVDLSLSCTLVPRSGEQAEGSPGGPGDSQGRKRRQTSMTDFYHSKRRLIFSKRKP

SEQ ID NO: 26 - DNA, 2175 bp, Homo sapiens
gttgtatatcagggccgcgctgagctgcgccagctgaggtgtgagcagctgccgaagtcagttccttgtggagccggagctgggcgcggattcgccgaggcaccgaggcactcagaggaggcgccatgtcagaaccggctggggatgtccgtcagaacccatgcggcagcaaggcctgccgccgcctcttcggcccagtggacagcgagcagctgagccgcgactgtgatgcgctaatggcgggctgcatccaggaggcccgtgagcgatggaacttcgactttgtcaccgagacaccactggagggtgacttcgcctgggagcgtgtgcggggccttggcctgcccaagctctaccttcccacggggccccggcgaggccgggatgagttgggaggaggcaggcggcctggcacctcacctgctctgctgcaggggacagcagaggaagaccatgtggacctgtcactgtcttgtacccttgtgcctcgctcaggggagcaggctgaagggtccccaggtggacctggagactctcagggtcgaaaacggcggcagaccagcatgacagatttctaccactccaaacgccggctgatcttctccaagaggaagccctaatccgcccacaggaagcctgcagtcctggaagcgcgagggcctcaaaggcccgctctacatcttctgccttagtctcagtttgtgtgtcttaattattatttgtgttttaatttaaacacctcctcatgtacataccctggccgccccctgccccccagcctctggcattagaattatttaaacaaaaactaggcggttgaatgagaggttcctaagagtgctgggcatttttattttatgaaatactatttaaagcctcctcatcccgtgttctccttttcctctctcccggaggttgggtgggccggcttcatgccagctacttcctcctccccacttgtccgctgggtggtaccctctggaggggtgtggctccttcccatcgctgtcacaggcggttatgaaattcaccccctttcctggacactcagacctgaattctttttcatttgagaagtaaacagatggcactttgaaggggcctcaccgagtgggggcatcatcaaaaactttggagtcccctcacctcctctaaggttgggcagggtgaccctgaagtgagcacagcctagggctgagctggggacctggtaccctcctggctcttgatacccccctctgtcttgtgaaggcagggggaaggtggggtcctggagcagaccaccccgcctgccctcatggcccctctgacctgcactggggagcccgtctcagtgttgagccttttccctctttggctcccctgtaccttttgaggagccccagctacccttcttctccagctgggctctgcaattcccctctgctgctgtccctcccccttgtcctttcccttcagtaccctctcagctccaggtggctctgaggtgcctgtcccacccccacccccagctcaatggactggaaggggaagggacacacaagaagaagggcaccctagttctacctcaggcagctcaagcagcgaccgccccctcctctagctgtgggggtgagggtcccatgtggtggcacaggcccccttgagtggggttatctctgtgttaggggtatatgatgggggagtagatctttctaggagggagacactggcccctcaaatcgtccagcgaccttcctcatccaccccatccctccccagttcattgcactttgattagcagcggaacaaggagtcagacattttaagatggtggcagtagaggctatggacagggcatgccacgtgggctcatatggggctgggagtagttgtctttcctggcactaacgttgagcccctggaggcactgaagtgcttagtgtacttggagtattggggtctgaccccaaacaccttccagctcctgtaacatactggcctggactgttttctctcggctccccatgtgtcctggttcccgtttctccacctagactgtaaacctctcgagggcagggaccacaccctgtactgttctgtgtctttcacagctcctcccacaatgctgaatatacagcaggtgctcaataaatgattcttagtgactttacttgtaaaaaaaaaaaaaaaaaa

SEQ ID NO: 27 - RNA, 9 nt, Artificial sequence
  Example of a sequences that can be used to form the shRNA loop
uucaagaga

SEQ ID NO: 28 - RNA, 9 nt, Artificial sequence
  Example of a sequences that can be used to form the shRNA loop
uuuguguag

SEQ ID NO: 29 - DNA, 19 bp, Artificial sequence
  Bcl-xl siRNA target sequence
cagggacagcatatcagag

SEQ ID NO: 30 - DNA, 19 bp, Artificial sequence
  Bcl-xl siRNA target sequence
ggagactagattgcctttg

SEQ ID NO: 31 - DNA, 21 bp, Artificial sequence
  Bcl-xl siRNA target sequence
ggcaggcgacgagtttgaact

SEQ ID NO: 32 - DNA, 21 bp, Artificial sequence
  Bcl-w siRNA target sequence
ctcggtcctgcgattattaat

SEQ ID NO: 33 - DNA, 20 bp, Artificial sequence
  Bcl-w siRNA target sequence
aagtgcaggagtggatggtg

SEQ ID NO: 34 - DNA, 21 bp, Artificial sequence
  Bcl-w siRNA target sequence
aagggttatgtctgtggagct

SEQ ID NO: 35 - DNA, 20 bp, Artificial sequence
  CDKN1A - cyclin-dependent kinase inhibitor 1A (p21, Cip1) siRNA target sequence
cttcgactttgtcaccgaga

SEQ ID NO: 36 - DNA, 21 bp, Artificial sequence
  CDKN1A - cyclin-dependent kinase inhibitor 1A (p21, Cip1) siRNA target sequence
cagtttgtgtgtcttaattat

SEQ ID NO: 37 - DNA, 21 bp, Artificial sequence
  CDKN1A - cyclin-dependent kinase inhibitor 1A (p21, Cip1) siRNA target sequence
ctggcattagaattatttaaa